(19) United States
(12) Patent Application Publication
(10) Pub. No.: US 2019/0038575 A1
(43) Pub. Date: Feb. 7, 2019

(54) Methods for Protecting Cells During Cancer Therapy

(71) Applicant: Applied Biology, Inc., Irvine, CA (US)

(72) Inventors: Ofer A. Goren, Irvine, CA (US); John McCoy, Irvine, CA (US)

(21) Appl. No.: 16/051,885

(22) Filed: Aug. 1, 2018

Related U.S. Application Data
(60) Provisional application No. 62/540,906, filed on Aug. 3, 2017.

(51) Int. Cl. A61K 31/137; A61Q 5/00; A61Q 7/00; A61P 17/14; A61K 9/00; A61K 47/10; A61K 47/26

(52) U.S. Cl. CPC A61K 31/137; A61Q 5/002; A61Q 7/00; A61K 47/26; A61K 9/0014; A61K 47/10; A61P 17/14

(57) ABSTRACT

Disclosed herein are compositions including a topical agent that may be used to reduce and/or inhibit the effects of chemotherapy and/or radiotherapy, which can include reducing and/or inhibiting the effects of hair loss and/or hair shedding due to chemotherapy and/or radiotherapy. The topical agent can be a weak acting alpha-1 adrenergic receptor agonist such as synephrine. Some embodiments of the composition can include a weak acting alpha-1 adrenergic receptor agonist with a modulating agent to modulate deposition, mode of action, and/or metabolism of a chemotherapeutic agent and/or a reactive oxygen species agent.

### CROSS-REFERENCE TO RELATED APPLICATIONS

This patent application is related to and claims the benefit of U.S. Provisional Application Ser. No. 62/540,906 filed on Aug. 3, 2017, the entire contents of which is incorporated herein by reference.

### TECHNICAL FIELD

The present invention relates to methods of treating or preventing forms of alopecia by topical applications of compositions that can include a weak acting alpha-1 adrenergic receptor agonist (e.g., synephrine). Some methods can include use of compositions to reduce and/or inhibit perfusion, which may facilitate treatment and/or prophylaxis of chemotherapy induced alopecia.

### BACKGROUND

Chemotherapy induced alopecia (“CIA”) can be a frequent adverse event present in patients undergoing oncological treatment, with an estimated incidence of 65% (see, Trueb 2009). CIA can have serious consequences to cancer patients' survivability. For example, 8% of women reject chemotherapy due to the potential risk of developing CIA. In addition, the psychological burden of CIA can lower self-esteem, body image, and quality of life (see, McGarvey 2001). A prophylactic treatment for CIA may be of significant clinical benefit to patients undergoing oncological treatment.

Effective chemotherapeutic agents can differentially affect rapidly dividing cells, e.g., cancer cells. Unfortunately, other rapidly dividing but noncancerous cells, such as, stem cells in the hair follicle niche, can also be affected. As a result, a significant number of patients undergoing chemotherapy may develop CIA. Some strategies for inhibiting CIA have been previously explored.

In 2017, the U.S. Food and Drug Administration (“FDA”) approved a scalp cooling system for reducing hair loss in breast cancer patients undergoing chemotherapy. Scalp cooling has been proven to effectively reduce the incidence of CIA. In a study of scalp cooling (see, Nangia et al. 2017), hair preservation was observed in approximately 50% of women that used the scalp cooling device; none of the women in the control group preserved hair. While scalp cooling as a prophylactic treatment for CIA is effective in some patients, it is time consuming, often uncomfortable, and expensive. Additionally, the procedure is not widely available.

Scalp cooling during chemotherapy sessions, has two proposed mechanisms for reducing CIA. First, scalp cooling induces cutaneous vasoconstriction resulting in reduced uptake of chemotherapeutic agents by hair follicles (see, FDA press release). Additionally, it has been proposed that the metabolism of chemotherapeutic agents or other important intermediates is reduced in hair follicle cells at lowered temperatures.

Other means of achieving the benefits of scalp cooling have been explored. Soref and Fahl (2015), demonstrated that topically applied vasoconstrictors reduced the reach of chemotherapeutic agents into the hair follicle niche and subsequently reduced the likelihood of developing CIA in rodents. Additionally, U.S. Patent Publication No. 20160136113 describes the use of specific vasoconstrictors to provide protection against the adverse effects, e.g., alopecia, mucositis or dermatitis, induced by chemotherapy or radiotherapy.

Additionally, other modes of modulating the deposition or metabolism of chemotherapy agents locally have been proposed. These agents can act on a specific chemotherapeutic agent or class of chemotherapeutic agents and could be applied locally, e.g., to the scalp, to stop chemotherapeutic agents from acting on the area of application. Similarly, other compounds have been proposed that inhibit CIA by different mechanisms. Many of these agents arrest cell growth in stages that are not vulnerable to chemotherapy. A few examples of such agents are provided in the table below.

TABLE 1
Examples of Agents That Arrest Cell Growth in Stages

| CIA Reducing | | | |
|---|---|---|---|
| Compound | Mode of Action | Chemotherapy | Reference |
| MAD11 | Inhibition (direct | doxorubicin and other | Balsari et al., |
| monoclonal | binding) of | anthracyclines | 1994 |
| antibody (MAb) | chemotherapeutic | | |
| | agent | | |
| Cyclosporine A | Inhibition of Go to | cyclophosphamide, cytosine | Hussein et al., |
| | G1 | arabinoside and etoposide | 1995 |
| N-acetyl cysteine | Antioxidant | cyclophosphamide | Wang et al., |
| | | | 2006 |
| alpha-tocopherol, | Antioxidant | doxorubicin | Batchelor, |
| beta- tocopherol, | | | 2001 |
| gamma- | | | |
| tocopherol, delta- | | | |
| tocopherol | | | |
| Calcitriol | Inhibition of Go to | cyclophosphamide, etoposide, | Jimenez and |
| | G1 | cyclophosphamide, | Yunis, 1996 |
| | | doxorubicin, and paclitaxel | |
| CDK2 inhibitor | Inhibition of G1 to | etoposide, 5-fluorouracil, | Davis et al., |
| | S | taxol, cisplatin, and | 2001 |
| | | doxorubicin | |
| 2,2′-methylenebis | Inhibition of | etoposide | Tsuda et al., |
| | Capsase-3 | | 2001 |
| Antioxidants | Reduction of ROS | Radiation Therapy | |
| Antioxidants | Reduction of ROS | anthracyclines (e.g., | Conklin, 2004 |
| | | doxorubicin, epirubicin, and | |
| | | daunorubicin), alkylating | |
| | | agents (e.g., | |
| | | cyclophosphamide), platinum | |
| | | coordination complexes (e.g., | |
| | | cisplatin, carboplatin, and | |
| | | oxaliplatin), and | |
| | | epipodophyllotoxins (e.g., | |
| | | etoposide) | |
| Dexamethasone | Up regulation of | docetaxel, paclitaxel | De Weger et |
| | CYP3A | | al., 2014 |
| Praeruptorin C | Up regulation of | taxanes | Huang et al., |
| | CYP3A | | 2013 |
| Rifampin, | Up regulation of | taxanes | |
| Carbamezapine, | CYP3A | | |
| Phenytoin, | | | |
| Phenobarbital, | | | |
| Corticosteroids, | | | |
| St. John's Wart | | | |
| Calcitriol-analogs | | cyclophosphamide | Schilli et al., |
| | | | 1998 |

The present invention concerns methods of treatment and/or prophylaxis of forms of alopecia by topical administration of a composition including a topical agent. In certain embodiments the topical agent reduces and or inhibits the effects of cancer treatment (e.g., chemotherapy, radiotherapy, etc.) which can include reducing and/or inhibiting the effects of hair loss and/or hair shedding due to chemotherapy and/or radiotherapy. The topical agent can be synephrine. Some embodiments of the composition can include a topical active agent with a modulating agent, the modulating agent being configured to modulate deposition, mode of action, and/or metabolism of a chemotherapeutic agent and/or a reactive oxygen species (“ROS”) agent. Some embodiments of the composition can be configured to induce hypoxia in rapidly dividing cells. Some embodiments of the composition can include a topical agent and/or a modulating agent configured to up-regulate CYP3A.

Embodiments can include an applicator kit. The applicator kit may include a spray device and/or a hair care product, such as a shampoo, conditioner, oil, etc. The application kit may include a container containing an embodiment of the composition and an applicator configured to test a region of the scalp of a person for perfusion.

In at least one embodiment, a method of treatment and/or prophylaxis of forms of alopecia can include applying a composition topically to a scalp of a person before, during, and/or after undergoing chemotherapy and/or radiation therapy. The composition may include a therapeutically effective amount of a weak acting alpha-1 adrenergic receptor agonist. Some embodiments can include applying the composition to the person so as to induce hypoxia in local tissue. Some embodiments can include applying the composition to the person so that amounts of chemotherapeutic agents and/or reactive oxygen species agents delivered to a hair follicle is limited. Some embodiments can include applying the composition to maintain a therapeutically effective amount for a prolonged period of time. The prolonged period of time can be within a range from 1 hour to 48 hours. In some embodiments, the weak acting agonist to alpha-1 adrenergic receptor is at least one of a selective alpha-1 adrenergic receptor agonist and an agonist having a low affinity for adrenergic receptors that are not alpha-1 adrenergic receptors. In some embodiments, the weak acting agonist to alpha-1 adrenergic receptors is synephrine. In some embodiments, the composition can further include a penetration enhancer. In some embodiments, the weak acting agonist to alpha-1 adrenergic receptors is synephrine and the penetration enhancer is pegylated caprylic/capric glycerides. In some embodiments, the composition can include about 30% to about 50% by weight of water, about 25% to about 40% by weight of synephrine, and about 5% to about 30% by weight of PEG-6 caprylic/capric glycerides. In some embodiments, the form of alopecia is chemotherapy induced alopecia and the chemotherapy is Taxane and/or Anthracycline-based chemotherapy. In some embodiments, the chemotherapy is used to treat stage I and/or stage II breast cancer patients.

In at least one embodiment, a method of treatment and/or prophylaxis of forms of alopecia can include applying a composition to a scalp of a person before, during, and/or after undergoing chemotherapy and/or radiation therapy, the composition comprising a therapeutically effective amount of a weak acting alpha-1 adrenergic receptor agonist and a modulating agent, wherein the modulating agent is configured to modulate deposition, mode of action, and/or metabolism of a chemotherapeutic agent and/or a reactive oxygen species agent. In some embodiments, the form of alopecia is chemotherapy induced alopecia and the chemotherapy is Taxane and/or Anthracycline-based chemotherapy. In some embodiments, the chemotherapy is used to treat stage I and/or stage II breast cancer patients.

In at least one embodiment, a method of treatment and/or prophylaxis of forms of alopecia can include applying a composition to a scalp of a person before, during, and/or after undergoing chemotherapy and/or radiation therapy, the composition comprising a therapeutically effective amount of a topical agent, wherein the topical agent is configured to up-regulate CYP3A. In some embodiments, the form of alopecia is chemotherapy induced alopecia and the chemotherapy is Taxane and/or Anthracycline-based chemotherapy. In some embodiments, the chemotherapy is used to treat stage I and/or stage II breast cancer patients.

In at least one embodiment, a method of treatment and/or prophylaxis of forms of alopecia can include applying a composition to a scalp of a person before, during, and/or after undergoing chemotherapy and/or radiation therapy, the composition comprising a therapeutically effective amount of a weak acting alpha-1 adrenergic receptor agonist and a modulating agent, the modulating agent configured to up-regulate CYP3A. In some embodiments, the form of alopecia is chemotherapy induced alopecia and the chemotherapy is Taxane and/or Anthracycline-based chemotherapy. In some embodiments, the chemotherapy is used to treat stage I and/or stage II breast cancer patients.

In at least one embodiment, a kit for treatment and/or prophylaxis of chemotherapy induced alopecia can include a container containing weak acting alpha-1 adrenergic receptor agonist. The kit may further include at least one of: an applicator configured to test a region of the scalp of a person for perfusion; and a disposable tip configured for use with a perfusion testing device.

In at least one embodiment, a kit for treatment and/or prophylaxis of forms of alopecia can include a container containing a weak acting alpha-1 adrenergic receptor agonist and a modulating agent, wherein the modulating agent is configured to modulate deposition, mode of action, and/or metabolism of a chemotherapeutic agent and/or a reactive oxygen species agent. The kit may further include at least one of: an applicator configured to test a region of the scalp of a person for perfusion; and a disposable tip configured for use with a perfusion testing device.

In at least one embodiment, a kit for treatment and/or prophylaxis of forms of alopecia can include a container containing a topical agent. The topical agent can be configured to up-regulate CYP3A. The kit may further include at least one of: an applicator configured to test a region of the scalp of a person for perfusion; and a disposable tip configured for use with a perfusion testing device.

In at least one embodiment, a kit for treatment and/or prophylaxis of forms of alopecia can include a container containing a weak acting alpha-1 adrenergic receptor agonist and a modulating agent. The modulating agent can be configured to up-regulate CYP3A. The kit may further include at least one of: an applicator configured to test a region of the scalp of a person for perfusion; and a disposable tip configured for use with a perfusion testing device.

In at least one embodiment, a hair care product for treatment and/or prophylaxis of forms of alopecia can include a pump spray container containing a weak acting A1AR agonist.

In at least one embodiment, a hair care product for treatment and/or prophylaxis of forms of alopecia can include a pump spray container containing a weak acting alpha-1 adrenergic receptor agonist and a modulating agent. The modulating agent can be configured to modulate deposition, mode of action, and/or metabolism of a chemotherapeutic agent and/or a reactive oxygen species agent.

In at least one embodiment, a hair care product for treatment and/or prophylaxis of forms of induced alopecia can include a pump spray container containing a topical agent. The topical agent can be configured to up-regulate CYP3A.

In at least one embodiment, a hair care product for treatment and/or prophylaxis of forms of alopecia can include a pump spray container containing a weak acting alpha-1 adrenergic receptor agonist and a modulating agent. The modulating agent can be configured to up-regulate CYP3A.

In at least one embodiment, a method of reducing and/or inhibiting perfusion of an anti-cancer agent into noncancerous organ or tissue or development of forms of alopecia in a person can include applying a therapeutically effective amount of a composition to the scalp of the person before, during, and/or after undergoing cancer treatment, the composition including about 30-40% synephrine by weight, water, PEG-6 caprylic/capric glycerides, and polysorbate. In at least one embodiment, the composition can further include a preservative, niacinamide, panthenol, sodium metabisulfite, phenoxyethanol ethylhexylglycerin and 1,3-propanediol.

In at least one embodiment, a method of treatment and/or prophylaxis of forms of alopecia can include applying an amount of a first composition to at least a portion of a scalp of a person before, during, and/or after chemotherapy and/or radiation therapy. The method can further include diagnosing response with a scalp perfusion test to determine if perfusion is reduced by a predetermined amount. If perfusion is reduced by the predetermined amount, the method may include applying a therapeutic effective amount of the first composition for treatment and/or prophylaxis of forms of alopecia. If perfusion is not reduced by the predetermined amount, the method may further include applying a second composition to at least a portion of the scalp of the person. The method may further include diagnosing response with a scalp perfusion test to determine if perfusion is reduced by a predetermined amount after application of the second composition. If perfusion is reduced by the predetermined amount, the method may further include applying a therapeutic effective amount of the second composition for treatment and/or prophylaxis of forms of alopecia. The first composition may include a concentration of a weak acting alpha-1 adrenergic receptor agonist that is less than a concentration of the weak acting alpha-1 adrenergic receptor agonist in the second composition.

In at least one embodiment, method of treatment and/or prophylaxis of forms of alopecia can include applying an amount of a first composition to at least a portion of a scalp of a person before, during, and/or after chemotherapy and/or radiation therapy. The method may further include diagnosing response with a scalp perfusion test to determine if perfusion is reduced by a predetermined amount. If perfusion is reduced by the predetermined amount, the method may further include applying a therapeutic effective amount of the first composition for treatment and/or prophylaxis of forms of alopecia. If perfusion is not reduced by the predetermined amount, the method may further include applying a second composition to at least a portion of the scalp of the person. The method may further include diagnosing response with a scalp perfusion test to determine if perfusion is reduced by a predetermined amount after application of the second composition. If perfusion is reduced by the predetermined amount, the method may further include applying a therapeutic effective amount of the second composition for treatment and/or prophylaxis of forms of alopecia. The first composition may include a concentration of a weak acting alpha-1 adrenergic receptor agonist and a modulating agent that is less than a concentration of the weak acting alpha-1 adrenergic receptor agonist and the modulating agent in the second composition. The modulating agent may be configured to modulate deposition, mode of action, and/or metabolism of a chemotherapeutic agent and/or a reactive oxygen species agent.

In at least one embodiment, a method of treatment and/or prophylaxis of forms of alopecia can include applying an amount of a first composition to at least a portion of a scalp of a person before, during, and/or after chemotherapy and/or radiation therapy. The method may further include diagnosing response with a scalp perfusion test to determine if perfusion is reduced by a predetermined amount. If perfusion is reduced by the predetermined amount, the method may further include applying a therapeutic effective amount of the first composition for treatment and/or prophylaxis of forms of alopecia. If perfusion is not reduced by the predetermined amount, the method may further include applying a second composition to at least a portion of the scalp of the person. The method may further include diagnosing response with a scalp perfusion test to determine if perfusion is reduced by a predetermined amount after application of the second composition. If perfusion is reduced by the predetermined amount, the method may further include applying a therapeutic effective amount of the second composition for treatment and/or prophylaxis of forms of alopecia. The first composition may include a concentration of a topical agent that is less than a concentration of the topical agent in the second composition. The topical agent may be configured to up-regulate CYP3A.

In at least one embodiment, a method of treatment and/or prophylaxis of forms of alopecia can include applying an amount of a first composition to at least a portion of a scalp of a person before, during, and/or after chemotherapy and/or radiation therapy. The method may further include diagnosing response with a scalp perfusion test to determine if perfusion is reduced by a predetermined amount. If perfusion is reduced by the predetermined amount, the method may further include applying a therapeutic effective amount of the first composition for treatment and/or prophylaxis of forms of alopecia. If perfusion is not reduced by the predetermined amount, the method may further include applying a second composition to at least a portion of the scalp of the person. The method may further include diagnosing response with a scalp perfusion test to determine if perfusion is reduced by a predetermined amount after application of the second composition. If perfusion is reduced by the predetermined amount, the method may further include applying a therapeutic effective amount of the second composition for treatment and/or prophylaxis of forms of alopecia. The first composition may include a concentration of a weak acting alpha-1 adrenergic receptor agonist and a modulating agent that is less than a concentration of the weak acting alpha-1 adrenergic receptor agonist and the modulating agent in the second composition. The modulating agent may be configured to up-regulate CYP3A.

### DETAILED DESCRIPTION

In certain embodiments, the topical active agent may be used to reduce and/or inhibit undesired side-effects of chemotherapy and/or radiotherapy, which can include reducing and/or inhibiting hair loss and/or hair shedding due to chemotherapy and/or radiotherapy. In some embodiments, the inventive methods can be used to reduce and/or inhibit perfusion of a chemotherapy agent to an organ or tissue that is noncancerous (for example skin epithelial cells), alopecia, and/or development of CIA. The topical active agent preferably is a weak acting alpha-1 adrenergic receptor (“A1AR”) agonist, which may be a selective A1AR agonist or an agonist having a low affinity for all other adrenergic receptors. In certain embodiments, the topical active agent is a modified strong acting A1AR agonist (modified to be weak-acting in vivo) or an alpha-2 adrenergic receptor (“A2AR”) agonist that acts as a weak A1AR agonist. It is contemplated for the topical active agent to be a weaker agonist for all receptors other than the alpha-1 adrenergic receptor. Some topical active agents can include indanidine, octopamine, synephrine, p-octopamine, p-synephrine, phenethylamine (PEA), also known as β-phenylethylamine (β-PEA), and 2-phenylethan-1-amine.

Some embodiments of the composition can include other active agents. These may or may not be weak acting A1AR agonists. Other active agents can include vasoconstrictors such as, without limitation, 25I-NBOMe, amphetamines, antihistamines, caffeine, phenylephrine, propylhexedrine, pseudoephedrine, etc. In certain embodiments, the other active agents are topically active and metabolize or slow the effects of a chemotherapeutic agent in the local area where applied.

Some embodiments of the invention involve co-administration of a topical active agent with a modulating agent, the modulating agent being configured to modulate deposition, mode of action, and/or metabolism of a chemotherapeutic agent and/or a ROS agent. Some embodiments of the invention involve methods to induce hypoxia in rapidly dividing cells. In some embodiments the invention involves administering a topical active agent such as a disclosed herein in combination with administration of a modulating agent configured to up-regulate CYP3A.

In at least one embodiment, the inventive methods and compositions are for topical application. For example, methods can include reducing and/or inhibiting perfusion of a chemotherapy agent to an organ or tissue that is noncancerous, alopecia, and/or CIA by applying a therapeutically effective amount of an embodiment of the composition to the skin (e.g., the scalp) of a person. This can include applying the therapeutic effective amount of an embodiment of the composition to the scalp of a person before, during, and/or after the person undergoes cancer treatment (e.g., receives chemotherapy and/or radiotherapy).

The active agents may be applied in the form of a composition that is a solution, an emulsion, and/or a gel. Some embodiments can include a kit. The kit can include an embodiment of the composition and other ingredient (e.g., shampoo). In some embodiments, the kit can include a container containing an embodiment of the composition and an applicator configured to test a region of the scalp of a person for perfusion. Other embodiments of the kit can include a container containing an embodiment of the composition and a disposable device (e.g., a tip) that can be used in conjunction with an applicator, the applicator being configured to test a region of the scalp of a person for perfusion. The applicator in this embodiment may or may not be part of the kit. Some embodiments can include a hair care product. The hair care product can include a pump spray container containing an embodiment of the composition. Some embodiments can include a pharmaceutical preparation. The pharmaceutical preparation can include an embodiment of the composition in a pharmaceutically-acceptable topical delivery vehicle suitable for topically delivering the composition.

Topical active agents according to the present invention include, without limitation, weak acting A1AR agonists. Preferably, a composition comprising a high concentration of a weak acting A1AR agonist is used. For example, embodiments of the composition can include high concentrations of a weak acting A1AR agonist and/or be formulated to allow for sufficient penetration of the weak acting A1AR agonist into the skin to provide effective amounts of the weak acting A1AR agonist at the hair follicle, such as by formulating with effective penetration enhancers. Without being in any way limited by theory, it is believed that a topical composition having a weak acting A1AR agonist may mediate local vasoconstriction through the alpha-1 adrenergic receptors in the smooth muscle of blood vessels.

Synephrine is an example of a weak acting A1AR agonist. In one embodiment, the invention involves application of a composition including synephrine to treat or prevent CIA. Synephrine is preferred because synephrine is unlikely to be distributed systemically in a high enough dosage to affect blood flow in other tissues, i.e., the synephrine composition can act topically to reduce blood flow to the hair follicles but will not interfere with delivery of chemotherapeutic agents to cancerous tissues. As will be explained herein, other weak acting A1AR agonists can be used.

Conventional methods of treatment may include use of compositions that do not contain a weak acting A1AR agonists and/or agonists having a low affinity for all other adrenergic receptors. Furthermore, conventional methods may not use weak acting A1AR agonists (or agonists having a low affinity for all other adrenergic receptors) as vasoconstrictors. Instead, conventional compositions may use strong or potent A1AR agonists as vasoconstrictors. With conventional compositions (e.g., those including potent A1AR agonists, such as norepinephrine or phenylephrine, for example), large concentrations are generally needed to penetrate the skin. For example, with phenylephrine, a 1% solution is used for injection, while a 10% solution is required to penetrate the eye for ophthalmic use. The surface of the scalp is quite large (600 cm²), and thus a large total dosage of a composition may be needed to observe a local effect. After the local effect of composition is achieved, excess amounts of the potent alpha-1 adrenergic receptor agonist may enter into the blood stream and cause systemic effects, such as undesirable cardiovascular effects that are measured by increase in blood pressure, for example. Thus, it is believed that strong or potent A1AR agonists can present a significant risk for adverse events when applied to large surface area such as the scalp. For instance, applications of phenylephrine, even at 2.5%-10% may yield 0.25 g to 0.5 g phenylephrine or over 200 mg of phenylephrine (provided 5 mL is used to cover the surface of the entire scalp). These amounts are in excess of prescription approved eye drops (e.g., maximum of 20.5 mg) or prescription approved oral forms (e.g., maximum of 20 mg). Such amounts can present a significant risk of cardiac events, as well as for pupil dilation if it enters the eye.

Some embodiments of the inventive methods disclosed herein, however, can use compositions formulated to allow applications of a large concentration of a weak acting A1AR agonist or agonists having a low affinity for all other adrenergic receptors. Weak acting A1AR agonists, such as synephrine for example, has low binding affinity to the alpha-1 receptor, as compared to more potent vasoconstrictors. Weak acting A1AR agonists can include agonists exhibiting potency approximately 150 times less than that of phenylephrine for human A1AR. A high concentration of the synephrine molecule can facilitate dermal penetration via a large entropic contribution to the patrician function (e.g., facilitate penetration through the stratum corneum), yet once in the bloodstream, the concentration of synephrine is diluted. This dilution, and the fact that it is a weak acting A1AR agonist, can result in avoidance or reduction of systemic effects such as increased blood pressure likely with other active agents. In addition, the mechanism of action for weak acting A1AR agonists may not be limited to vasoconstriction or hypoxia in the local tissue. The mechanism of action including both vasoconstriction and induced hypoxia can result in reduced metabolism of a chemotherapeutic agent, which may further facilitate the reduction or prevention of systemic effects.

Use of weak acting A1AR agonists or agonists having a low affinity for all other adrenergic receptors, as described herein, can provide methods of treatment and/or prophylaxis that are a substantial improvement over conventional methods of use. Improvements can include enhancements in efficacy and/or safety. For example, safety tests were performed with embodiments of the composition using electrocardiogram (EKG) and blood pressure/heart rate (BP/HR) monitoring. No adverse events were found with topical application across the entire scalp of a composition including synephrine at up to 6 g (e.g., 5 mL of 37% synephrine HCL or 30% synephrine). Details of the study are described in the examples below.

Topical active agents for use in the present invention include weak acting A1AR agonists such as without limitation, indanidine, octopamine, synephrine, p-octopamine, p-synephrine, phenethylamine (PEA), also known as β-phenylethylamine (β-PEA), and 2-phenylethan-1-amine. In addition, some strong acting A1AR agonists may be modified to act as weak A1AR agonists. For example, synephrine (a weak A1AR agonist) and phenylephrine (a strong A1AR agonist) are structurally similar. Phenylephrine, however, has the hydroxyl in the meta position, whereas synephrine has the hydroxyl in the para position. The compounds have significantly different biological properties. Thus, it is contemplated that modifications can be made to some strong acting A1ARagonists to generate a weak acting A1AR agonist, or at least a weaker acting A1AR agonist. Additionally, derivatives of A1AR agonists can be utilized including derivatives of the compounds mentioned above. In other embodiments, a prodrug that is activated to become an A1AR agonist can be utilized. A particular prodrug can be activated by endogenous enzymes in the scalp such as Caspase-1 when follicular inflammation is present, e.g., at the location of application of a hair extension. Moreover, some A2AR agonists can act as weak A1AR agonists. Some of these A2AR agonists that are useful in the present invention can include, but are not limited to, clonidine, guanfacine, guanabenz, guanoxabenz, guanethidine, xylazine, tizanidine, medetomidine, methyldopa, methylnorepinephrine, fadolmidine, and demedetomidine. Any one or combination of the above-identified agonists may be used in embodiments of the invention.

Preferably, the agonist used is a selective A1AR agonist (e.g., low binding affinity for alpha-1 receptors) and/or has a low affinity for all other adrenergic receptors (e.g., low affinity to adrenergic receptors that are not alpha-1 receptors). A weak A1AR agonist having a strong affinity to another receptor may be undesirable due to safety concerns. Furthermore, being a selective A1AR agonist and/or having a low affinity for all other adrenergic receptors can allow for the composition to be applied in high doses without significant side effects resulting from effects at other adrenergic receptors.

The present invention may be used in cancer therapy, such as without limitation cancer treatment (e.g., taxmen therapy) for breast cancer and colorectal cancer. However, the present invention can be applicable to most, if not all, types of cancers (e.g., solid organ tumor cancers, sarcomas, carcinoma, lymphomas, etc.) and associated cancer treatments. In at least one embodiment, the present invention can be used as a prophylactic treatment of CIA for patients undergoing cancer treatment with Taxane and/or Anthracycline-based chemotherapy. This can include stage I and/or stage II breast cancer patients.

In at least one variation, an embodiment of the invention involves administration of any one or combination of the weak acting A1AR agonists or the agonists having a low affinity for all other adrenergic receptors identified herein and exclude any one or combination of the following: epinephrine, phenylephrine, methoxamine, norepinephrine, zolmitriptan, tetrahydrozoline, naphazoline, prazosin, doxazosin, terazosin, alfuzosin, and tamsulosin.

In some embodiments, the present invention can include use of a weak acting A1AR agonist or an agonist having a low affinity for all other adrenergic receptors topically applied to the scalp. This may be done to reduce and/or inhibit perfusion. This may limit the amount of chemotherapeutic agents delivered to the hair follicle. Limiting the amount of chemotherapeutic agents delivered the hair follicle can reduce and/or inhibit CIA.

In some embodiments, the present invention can include use of a topical active agent in combination with a modulating agent topically applied to the scalp. The topical active agent can be a weak acting A1AR agonist, an agonist having a low affinity for all other adrenergic receptors, a strong acting A1AR agonist, or any combination thereof. The modulating agent can be configured to modulate deposition, mode of action, and/or metabolism of a chemotherapeutic agent. A modulating agent can include any one or combination of caffeine, MAD 11 monoclonal antibody (MAb), Cyclosporine A, N-acetyl cysteine, alpha-tocopherol, beta-tocopherol, gamma-tocopherol, delta-tocopherol, Calcitriol, CDK2 inhibitor, 2,2′-methylenebis, Antioxidants, Dexamethasone, Praeruptorin C, Rifampin, Carbamezapine, Phenytoin, Phenobarbital, Corticosteroids, St. John's Wart, and Calcitriol-analogs.

In some embodiments, the present invention can include use of a weak acting A1AR agonist or an agonist having a low affinity for all other adrenergic receptors topically applied to the scalp. This may be done to reduce and/or inhibit perfusion. This may limit the amount of ROS agents delivered to the hair follicle. Limiting ROS agents delivered to the hair follicle can reduce and/or inhibit CIA due to radiotherapy.

In some embodiments, the present invention can include use of a weak acting A1AR agonist or an agonist having a low affinity for all other adrenergic receptors topically applied to the scalp. This may be done to reduce and/or inhibit perfusion. This may limit the amount of ROS agents delivered to the hair follicle. Limiting ROS agents delivered to the hair follicle can reduce and/or inhibit CIA when ROS is the mode of action of a chemotherapeutic compound used for chemotherapy treatment.

In some embodiments, the present invention can include use of a weak acting A1AR agonist or an agonist having a low affinity for all other adrenergic receptors topically applied to the scalp. With some embodiments, the composition can be applied all at once. Some embodiments involve multiple applications of the composition. In a preferred embodiment, the composition is applied 10-30 minutes prior to chemotherapy and/or radiotherapy and at the end of chemotherapy and/or radiotherapy (e.g., about 2 hours after beginning the chemotherapy and/or radiotherapy). Some embodiments can include applying a therapeutically effective amount of an embodiment of the composition for a prolonged period of time, such as, without limitation, for 48, hours, 24 hours, 12 hours, 6 hours, 5 hours, 4 hours, 3 hours, 2 hours, or one hour, or any amount of time there-between. Some embodiments can include applying the composition repeatedly for the prolonged period of time (e.g., applying the composition every 30 minutes, one hour, 2 hours, 3 hours, 4 hours, etc.). This can include re-applying the composition for up to 48 hours before, during, and/or after undergoing chemotherapy and/or radiotherapy. In some embodiments, the composition can be applied before undergoing chemotherapy and/or radiotherapy treatment and then for a prolonged period of time after undergoing chemotherapy and/or radiotherapy. In some embodiments, the composition can be applied for a prolonged period of time before undergoing chemotherapy and/or radiotherapy treatment and then applied after undergoing chemotherapy and/or radiotherapy. In some embodiments, the composition can be applied for a prolonged period of time before undergoing chemotherapy and/or radiotherapy treatment and then applied for a prolonged period of time after undergoing chemotherapy and/or radiotherapy. Applying the composition for a prolonged period of time may induce hypoxia in local tissue. For example, this may induce hypoxia in rapidly dividing cells. Inducing hypoxia in rapidly dividing cells can reduce the effect of chemotherapeutic agents delivered to the hair follicle. Reducing the effect of chemotherapeutic agents delivered to the hair follicle can reduce the likelihood of developing CIA.

In some embodiments, the present invention can include use of a topical active agent in combination with a modulating agent topically applied to the scalp. The topical active agent can be a weak acting A1AR agonist, an agonist having a low affinity for all other adrenergic receptors, a strong acting A1AR agonist, or any combination thereof. The modulating agent can be configured to modulate deposition, mode of action, and/or metabolism of a ROS. This may be done to reduce and/or inhibit CIA due to radiotherapy. A modulating agent can include any one or combination of caffeine, MAD 11 monoclonal antibody (MAb), Cyclosporine A, N-acetyl cysteine, alpha-tocopherol, beta-tocopherol, gamma-tocopherol, delta-tocopherol, Calcitriol, CDK2 inhibitor, 2,2′-methylenebis, Antioxidants, Dexamethasone, Praeruptorin C, Rifampin, Carbamezapine, Phenytoin, Phenobarbital, Corticosteroids, St. John's Wart, and Calcitriol-analogs.

In some embodiments, the present invention can include use of a topical active agent in combination with a modulating agent topically applied to the scalp. The topical active agent can be a weak acting A1AR agonist, an agonist having a low affinity for all other adrenergic receptors, a strong acting A1AR agonist, or any combination thereof. The modulating agent can be configured to modulate deposition, mode of action, and/or metabolism of a ROS. This may be done to reduce and/or inhibit CIA when ROS is the mode of action of a chemotherapeutic compound used in a chemotherapy treatment. A modulating agent can include any one or combination of caffeine, MAD 11 monoclonal antibody (MAb), Cyclosporine A, N-acetyl cysteine, alpha-tocopherol, beta-tocopherol, gamma-tocopherol, delta-tocopherol, Calcitriol, CDK2 inhibitor, 2,2′-methylenebis, Antioxidants, Dexamethasone, Praeruptorin C, Rifampin, Carbamezapine, Phenytoin, Phenobarbital, Corticosteroids, St. John's Wart, and Calcitriol-analogs.

In some embodiments, the present invention can include use of a first topical agent and a second topical agent topically applied to the scalp, where the second topical agent is configured to up-regulate CYP3A. This can be done to reduce and/or inhibit CIA due to taxanes (e.g., docetaxel and paclitaxel) that may be used for treatment therapy. A second topical agent can include any one or combination of caffeine, MAD 11 monoclonal antibody (MAb), Cyclosporine A, N-acetyl cysteine, alpha-tocopherol, beta-tocopherol, gamma-tocopherol, delta-tocopherol, Calcitriol, CDK2 inhibitor, 2,2′-methylenebis, Antioxidants, Dexamethasone, Praeruptorin C, Rifampin, Carbamezapine, Phenytoin, Phenobarbital, Corticosteroids, St. John's Wart, and Calcitriol-analogs.

In some embodiments, the present invention can include use of a topical active agent and a modulating agent topically applied to the scalp, where the modulating agent is configured to up-regulate CYP3A. The topical active agent can be a weak acting A1AR agonist, an agonist having a low affinity for all other adrenergic receptors, a strong acting A1AR agonist, or any combination thereof. This can be done to reduce CIA due to taxanes (e.g., docetaxel and paclitaxel) that may be used for treatment therapy. A modulating agent can include any one or combination of caffeine, MAD 11 monoclonal antibody (MAb), Cyclosporine A, N-acetyl cysteine, alpha-tocopherol, beta-tocopherol, gamma-tocopherol, delta-tocopherol, Calcitriol, CDK2 inhibitor, 2,2′-methylenebis, Antioxidants, Dexamethasone, Praeruptorin C, Rifampin, Carbamezapine, Phenytoin, Phenobarbital, Corticosteroids, St. John's Wart, and Calcitriol-analogs.

The topical active agent as used herein can include synephrine, which may include bitter orange extract (extract of Citrus Aurantium L.). Embodiments may further relate to methods of preparing the compositions, methods of administering the compositions, methods of treatment or prevention of the effects of hair loss due to chemotherapy and/or radiotherapy by applying or administering therapeutically effective amounts of the compositions, and kits comprising the embodiments of the composition and at least one additional hair care product or container for administering the compositions.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims.

As used herein, the terms “prevent” or “prevention” and other derivatives of the words, when used in reference to alopecia, e.g., CIA, refer to a reduced likelihood of alopecia in an individual receiving a given treatment relative to that of a similar individual at risk for alopecia but not receiving that treatment. As such, the terms “prevent” and “prevention” encompass a treatment that results in a lesser degree of alopecia than would be otherwise expected for a given individual.

Efficacy for reducing hair loss, inhibiting hair loss, and prevention of alopecia can be established through controlled studies in which a subject is administered a treatment (e.g., a topical treatment) at one site likely to experience or exhibit hair loss or hair shedding but not at another site subjected to the same conditions. Under these circumstances, if the site receiving the topical treatment undergoes less hair loss over time relative to the untreated site, e.g., at least 5% less, at least 10% less, at least 15% less, at least 20% less, at least 25% less, at least 30% less, at least 35% less, at least 40% less, at least 45% less, at least 50% less or beyond, the treatment is effective for reducing hair loss. Determination of efficacy also may involve the measurement or detection of pilomotor stimulation, which can be performed, at its simplest, by observation of the area at the base of the hair shaft to determine whether the smooth muscle of the blood vessels has contracted.

As used herein, the terms “treat,” “treatment,” or “treating” refers to reversing, inhibiting, slowing down or stopping the progression or severity of a disease or condition, e.g., CIA or other form of alopecia. Treatment of alopecia, and particularly CIA, is generally “effective” if hair loss or hair shedding is slowed or stopped, or hair regrows at a faster rate than hair is lost. The methods of showing efficacy for prevention of alopecia discussed above are also applicable for showing efficacy of treatment of alopecia.

As used herein, the term “inhibit” or “inhibiting” in the context of hair loss or hair shedding means to reduce the amount of hair removed or hair that falls out, such as reducing the amount of hair shedding when a cosmetic procedure is applied to the hair.

As used herein the term “perfusion” refers to the passage of fluid through the circulatory system or lymphatic system to an organ or a tissue, usually referring to the delivery of blood to a capillary bed in tissue.

As used herein the term “comprising” or “comprises” is used in reference to compositions, methods, etc. refers to component(s) or method steps that are present in the method or composition, yet allows for the composition, method, etc. to also include unspecified elements.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Compositions of Synephrine

The compositions of the present disclosure can contain synephrine. A source of synephrine that may be used in the compositions of the technology described herein can be Citrus Aurantium, conventionally known as bitter orange, seville orange, sour orange, bigarade orange, or marmalade orange. Substantially all of the synephrine in bitter orange extract is in the R(−) enantiomeric form. The present disclosure encompasses formulations of synephrine in which the synephrine is present in the R(−) enantiomer, in the S (+) enantiomer, in mixtures of both R and S enantiomers, and as a racemic mixture of R and S enantiomers. Another source of synephrine can include synephrine HCl (synthetically prepared synephrine).

Some embodiments can include a racemic mixture. For example, a preferred embodiment may include a racemic mixture of R-(−)-synephrine and S-(+)-synephrine. This may be preferred because a racemic mixture is half as potent as a pure “active” enantiomer. Furthermore, a racemic mixture may be used to create a low affinity compound via selection of the “non-active” enantiomer. For example, if (−)oxymetazoline is active, (+)oxymetazoline may be used as a low affinity A1AR agonist.

“Bitter Orange Extract” as used herein is a product derived from Citrus Aurantium Amara (bitter orange) that contains about 6-99% by weight of R (−) synephrine. The extract may be of the peel powder.

The compositions according to the present disclosure can include synephrine and water. In one embodiment, the compositions further comprise a penetration enhancer, such as caprylic/capric glyceride, preferably PEG-6 caprylic/capric glyceride. Applicants have discovered that high concentrations of synephrine, such as without limitation of bitter orange extract, are difficult to formulate into stable compositions with water. Surprisingly, Applicants have found that formulations of synthetically prepared synephrine (synephrine HCl) can be produced with higher synephrine concentrations than compositions of synephrine that is obtained from bitter orange extract. Furthermore, the hair loss and hair shedding described can be addressed and inhibited by applying compositions in which a relatively high amount of synephrine is delivered through the dermis at the follicle to the base of the hair follicle. Compositions have been discovered that provide for high levels of synephrine to penetrate the dermis and reach the base of the hair follicle. In particular, the inventive compositions contain or comprise penetration enhancers, preferably caprylic/capric glyceride and derivatives thereof.

In one embodiment, the composition comprises between about 10% to about 60% by weight of synephrine and between about 10% and about 60% water by weight. In other embodiments, synephrine is present at about 10% to 20%, and water is present at about 10% to 20%. In other embodiments, synephrine is present at about 10% to 20% and water at about 10% to 30%; synephrine is present at about 20% to 30%, and water is present at about 10% to about 40%; synephrine is present at about 30% to 40%, and water is present at about 10% to about 50%; synephrine is present at about 40% to 50%, and water is present at about 20% to about 40%; and synephrine is present at about 30% to 50%, and water is present at about 10% to about 40%. For purposes of these weight ranges, the weight of synephrine is based on the weight of synephrine in free base form. Other forms, such as salt forms, of synephrine may be used, in which cases the weight percentage above reflects the equivalent weight of synephrine free base. Naturally sourced synephrine, such as that obtained from bitter orange extract, has a lower molecular weight (167.2) than a salt form of synephrine that is produced synthetically, synephrine HCl (203.7). As such, more of the salt form is needed than the freebase form to have the same concentration of the synephrine molecule in solution. For example, a 37% w/w synephrine HCl composition contains the equivalent synephrine content as a 32% w/w synephrine free base composition. As used herein, a 25-35% w/w synephrine composition encompasses a composition containing 37% synephrine HCl because its equivalent synephrine weight percentage is 30% w/w, which is within the range of 25-35%.

In a further embodiment, the composition further comprises caprylic/capric glyceride, such as PEGylated caprylic/capric glycerides, including without limitation PEG-6 caprylic/capric glyceride such as Acconon® CC-6 PEG-6 caprylic/capric glycerides (supplied by Abitec Corporation) and Tegosoft® GMC 6 PEG-6 caprylic/capric glycerides (supplied by Evonik Industries AG). In some embodiments, the composition comprises about 20% to about 40% by weight of water, about 25% to about 35% by weight of synephrine, and about 5% to about 40% by weight of PEGylated caprylic/capric glyceride. The composition may comprise at least one additional additive. In one embodiment, the composition comprises between about 10% to about 60% by weight of synephrine, between about 10% and about 60% by weight water, and between about 5% and about 30% by weight PEGylated caprylic/capric glyceride. In other embodiments, synephrine is present at about 10% to 20%, water is present at about 10% to 20%, and PEGylated caprylic/capric glyceride is present at about 5% to 20%. In other embodiments, synephrine is present at about 10% to 20%, water at about 10% to 30%, and PEGylated caprylic/capric glyceride at about 5% to 20%. In a further embodiment, synephrine is present at about 20% to 30%, water is present at about 10% to about 40% and PEGylated caprylic/capric glyceride is present at about 5% to 20%. In a further embodiment, synephrine is present at about 30% to 40%, water is present at about 10% to about 50%, and PEGylated caprylic/capric glyceride is present at about 5% to about 20%. In yet a further embodiment, synephrine is present at about 40% to 50%, water is present at about 20% to about 40%, and PEGylated caprylic/capric glyceride is present at about 5% to about 20%. In yet a further embodiment, synephrine is present at about 30% to 50%, water is present at about 10% to about 40%, and PEGylated caprylic/capric glyceride is present at about 5% to about 15%. For purposes of these weight ranges, if a product other than synephrine free base is used, the weight percentage corresponds to the equivalent weight percentage of synephrine in free base form. Typically, synthetically produced synephrine comprises S-(+)-synephrine and R-(−)-synephrine. Brown, C. M. et al., “Activities of octopamine and synephrine stereoisomers on alpha-adrenoceptors,” Br. J. Pharmacol. (1988), 93, 417-429 describes the activities of the stereoisomers of p-synephrine (referred to as synephrine herein) on postjunctional alpha1-alpha2-adrenoceptors. The potency of the (+) form was one to two orders of magnitude less than the (−) form on these two adrenoceptors. Accordingly, Applicants believe that R-(−)-synephrine has greater activity for the treatment and conditions disclosed herein than S-(+)-synephrine. However, using the commercial synephrine sources and the experiments described herein, an efficacy difference has not been noted experimentally. The synephrine used in the inventions disclosed herein is p-synephrine, and may be obtained from natural sources or synthetically produced, and may be a salt, solvate or hydrate of p-synephrine. The synephrine that may be used in the inventive compositions and methods herein includes without limitation bitter orange extract; a racemic mixture of R-(−)-synephrine and S-(+)-synephrine; synephrine that is greater than 40% R-(−)-synephrine and 60% or less by weight S-(+)-synephrine; synephrine that is greater than 50% R-(−)-synephrine and 50% or less by weight S-(+)-synephrine; synephrine that is greater than 60% R-(−)-synephrine and 40% or less by weight S-(+)-synephrine; synephrine that is greater than 70% R-(−)-synephrine and 30% or less by weight S-(+)-synephrine; synephrine that is greater than 90% R-(−)-synephrine and 10% or less by weight S-(+)-synephrine; synephrine that is greater than 95% R-(−)-synephrine; synephrine that is greater than 98% R-(−)-synephrine; synephrine that is substantially all R-(−)-synephrine; or any mixture of R-(−)-synephrine and S-(+)-synephrine.

In some embodiments of the present disclosure, the composition contains synephrine or synephrine HCl, each in any enantiomeric form or mixtures thereof, as a weight percentage based on the total weight of the composition of about 10% to about 60%, about 15% to about 60%, about 20% to about 60%, about 25% to about 60%, 30% to about 60%, about 10% to about 55%, about 10% to about 50%, about 15% to about 50%, about 20% to about 50%, about 25% to about 50%, about 30% to about 50%, about 35% to about 50%, about 40% to about 50%, about 45% to about 50%, about 10% to about 40%, about 15% to about 40%, about 20% to about 40%, about 25% to about 40%, about 15% to about 35%, about 20% to about 35%, about 25% to about 35%, about 20% to about 30%, about 30% to about 35%, about 20% to about 70%, about 25% to about 70%, about 30% to about 70%, about 35% to about 70%, about 40% to about 70%, about 45% to about 70%, about 50% to about 70%, about 55% to about 70%, about 20% to about 65%, about 25% to about 65%, about 30% to about 65%, about 35% to about 65%, about 40% to about 65%, about 45% to about 65%, about 50% to about 65%, or about 55% to about 65%. Other forms of synephrine, such as other salt forms than HCl, may be used. The appropriate weight ranges for other forms of synephrine should be determined based on the guidance above of weight ranges for synephrine and synephrine HCl.

The compositions of the present disclosure contain water, as a weight percentage based on the total weight of the composition, of about 60%, about 15% to about 60%, about 20% to about 60%, about 25% to about 60%, 30% to about 60%, about 10% to about 55%, about 10% to about 50%, about 15% to about 50%, about 20% to about 50%, about 25% to about 50%, about 30% to about 50%, about 35% to about 50%, about 40% to about 50%, about 45% to about 50%, about 10% to about 40%, about 15% to about 40%, about 20% to about 40%, about 25% to about 40%, about 15% to about 35%, about 20% to about 35%, about 25% to about 35%, about 20% to about 30%, about 30% to about 35%, about 20% to about 70%, about 25% to about 70%, about 30% to about 70%, about 35% to about 70%, about 40% to about 70%, about 45% to about 70%, about 50% to about 70%, about 55% to about 70%, about 20% to about 65%, about 25% to about 65%, about 30% to about 65%, about 35% to about 65%, about 40% to about 65%, about 45% to about 65%, about 50% to about 65%, or about 55% to about 65%. The composition may comprise about 10% to about 60%, about 15% to about 60%, about 20% to about 60%, about 25% to about 60%, 30% to about 60%, about 10% to about 55%, about 10% to about 50%, about 15% to about 50%, about 20% to about 50%, about 25% to about 50%, about 30% to about 50%, about 35% to about 50%, about 40% to about 50%, about 45% to about 50%, about 10% to about 40%, about 15% to about 40%, about 20% to about 40%, about 25% to about 40%, about 15% to about 35%, about 20% to about 35%, about 25% to about 35%, about 20% to about 30%, about 30% to about 35%, about 20% to about 70%, about 25% to about 70%, about 30% to about 70%, about 35% to about 70%, about 40% to about 70%, about 45% to about 70%, about 50% to about 70%, about 55% to about 70%, about 20% to about 65%, about 25% to about 65%, about 30% to about 65%, about 35% to about 65%, about 40% to about 65%, about 45% to about 65%, about 50% to about 65%, about 55% to about 65%, by weight of water.

Embodiments of the present disclosure can include compositions comprising water, synephrine, and a penetration enhancer. In some embodiments, the composition further comprises additives, such as components to improve the miscibility of the composition. In some embodiments, the composition further comprises at least one preservative.

A penetration enhancer or permeation enhancer is an agent used to increase the permeability of the stratum corneum (“SC”). The SC is the outer most layer of skin. Cornification of the SC makes it a good barrier to most water soluble molecules. Penetration enhancers typically disrupt the barrier function of the SC. After passage through the SC, molecules are free to diffuse into deeper tissues. A chemical penetration enhancer increases skin permeability by reversibly altering the physiochemical nature of the tissue to reduce its diffusional resistance. According to one or more embodiments of the present invention a penetration enhancer is incorporated into the composition. It is to be understood that components identified herein as penetration enhancers may have other roles in the formulation also, such as act as an emulsifying agent, secondary surfactant, or emollient.

Examples of penetration enhancers according to the present invention include: polyols, such as propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, and glycerol; sulfoxides, such as dimethylsulfoxide (DMSO), dimethylformanide, methyl dodecyl sulfoxide, dimethylacetamide; monooleate of ethoxylated glycerides (with 8 to 10 ethylene oxide units); azone (1-dodecylazacycloheptan-2-one), 2-(n-nonyl)-1,3-dioxolane; esters, such as isopropyl myristate/palmitate, ethyl acetate, butyl acetate, methyl proprionate, capric/caprylic triglycerides, PEG-6 capylic/capric glycerides, PEG-7 caprylic/capric glycerides, mixed decanoyl and octanoyl glycerides, octylmyristate, dodecyR-(−)-myristate; myristyl alcohol, lauryl alcohol, lauric acid, lauryl lactate ketones; amides, such as acetamide oleates such as triolein; various surfactants, such as sodium lauryl sulfate; various alkanoic acids such as caprylic acid; lactam compounds, such as azone; alkanols, such as oleyl alcohol; dialkylamino acetates, and mixtures thereof. Other penetration enhancers that may be used are cyclodextrins and related compounds. Cyclodextrins are structurally related cyclic oligomaltoses.

In the preferred embodiments, the penetration enhancer is PEG-6 capylic/capric glycerides, such as the products sold under the trade names ACCONON® CC-6 and Tegosoft® GMC 6. The composition may comprise about 4% to about 20%, about 5% to about 20%, about 5% to about 15%, about 6% to about 15%, about 7% to about 15%, about 8% to about 15%, about 9% to about 15%, about 10% to about 15%, about 11% to about 15%, about 12% to about 15%, about 5% to about 14%, about 6% to about 14%, about 7% to about 14%, about 8% to about 14%, about 9% to about 14%, about 5% to about 13%, about 6% to about 13%, about 7% to about 13%, about 8% to about 13%, about 9% to about 13%, about 5% to about 12%, about 6% to about 12%, about 7% to about 12%, about 8% to about 12%, about 9% to about 12%, about 10% to about 12%, about 5% to about 11%, about 6% to about 11%, about 7% to about 11%, about 8% to about 11%, about 9% to about 11%, about 10% to about 11%, about 5% to about 10%, about 6% to about 10%, about 7% to about 10%, about 8% to about 10%, about 9% to about 10%, or about 30% to about 35% by weight of a penetration enhancer such as caprylic/capric glycerides or PEGylated caprylic/capric glycerides, preferably PEG-6 caprylic/capric glycerides, based on the total weight of the composition.

Compositions comprising about 10% to about 20% by weight of synephrine and about 35% to about 60% by weight of water may comprise about 20% to about 40%, about 25% to about 40%, about 30% to about 40%, about 35% to about 40%, about 20% to about 35%, about 25% to about 35%, about 30% to about 35%, about 20% to about 30%, about 25% to about 30%, or about 20% to about 25% by weight of penetration enhancer such as caprylic/capric glycerides or PEGylated caprylic/capric glycerides, preferably PEG-6 caprylic/capric glycerides.

Compositions comprising about 15% to about 25% by weight of synephrine and about 30% to about 55% by weight of water may comprise about 15% to about 40%, about 20% to about 40%, about 25% to about 40%, about 30% to about 40%, about 35% to about 40%, about 15% to about 35%, 20% to about 35%, about 25% to about 35%, about 30% to about 35%, about 15% to about 30%, about 20% to about 30%, about 25% to about 30%, about 15% to about 25%, about 20% to about 25%, or about 15% to about 20% by weight of penetration enhancer such as caprylic/capric glycerides or PEGylated caprylic/capric glycerides, preferably PEG-6 caprylic/capric glycerides.

Compositions comprising about 20% to about 30% by weight of synephrine and about 25% to about 50% by weight of water may comprise about 10% to about 40%, 15% to about 40%, about 20% to about 40%, about 25% to about 40%, about 30% to about 40%, about 35% to about 40%, about 10% to about 35%, about 15% to about 35%, 20% to about 35%, about 25% to about 35%, about 30% to about 35%, about 10% to about 30%, about 15% to about 30%, about 20% to about 30%, about 25% to about 30%, about 10% to about 25%, about 15% to about 25%, about 20% to about 25%, about 10% to about 20%, about 15% to about 20%, or about 10% to about 15% by weight of penetration enhancer such as caprylic/capric glycerides or PEGylated caprylic/capric glycerides, preferably PEG-6 caprylic/capric glycerides.

Compositions comprising about 30% to about 40% by weight of synephrine and about 40% to about 55% by weight of water may comprise about 5% to about 35%, about 10% to about 35%, about 15% to about 35%, 20% to about 35%, about 25% to about 35%, about 30% to about 35%, about 5% to about 30%, about 10% to about 30%, about 15% to about 30%, about 20% to about 30%, about 25% to about 30%, about 5% to about 25%, about 10% to about 25%, about 15% to about 25%, about 20% to about 25%, about 5% to about 20%, about 10% to about 20%, about 15% to about 20%, about 3% to about 15%, about 4% to about 15%, about 5% to about 15%, about 6% to about 15%, 7% to about 15%, about 8% to about 15%, about 9% to about 15%, about 10% to about 15%, about 11% to about 15%, about 12% to about 15%, about 13% to about 15%, about 14% to about 15%, about 3% to about 14%, about 4% to about 14%, about 5% to about 14%, about 6% to about 14%, about 7% to about 14%, about 8% to about 14%, about 9% to about 14%, about 10% to about 14%, about 11% to about 14%, about 12% to about 14%, about 13% to about 14%, about 3% to about 13%, about 4% to about 13%, about 5% to about 13%, about 6% to about 13%, about 7% to about 13%, about 8% to about 13%, about 9% to about 13%, about 3% to about 12%, about 4% to about 12%, about 5% to about 12%, about 6% to about 12%, about 7% to about 12%, about 8% to about 12%, about 9% to about 12%, about 10% to about 12%, about 11% to about 12%, about 3% to about 11%, about 4% to about 11%, about 5% to about 11%, about 6% to about 11%, about 7% to about 11%, about 8% to about 11%, about 9% to about 11%, about 10% to about 11%, about 3% to about 10%, about 4% to about 10%, about 5% to about 10%, about 6% to about 10%, about 7% to about 10%, about 8% to about 10%, about 9% to about 10%, about 3% to about 9%, about 4% to about 9%, about 5% to about 9%, about 6% to about 9%, about 7% to about 9%, about 8% to about 9%, about 3% to about 8%, about 4% to about 8%, about 5% to about 8%, about 6% to about 8%, about 7% to about 8%, about 3% to about 7%, about 4% to about 7%, about 5% to about 7%, about 6% to about 7%, about 3% to about 6%, about 4% to about 6%, about 5% to about 6%, about 3% to about 5%, about 4% to about 5%, or about 3% to about 4% by weight of penetration enhancer such as caprylic/capric glycerides or PEGylated caprylic/capric glycerides, preferably PEG-6 caprylic/capric glycerides.

Compositions comprising about 30% to about 40% by weight of synephrine and about 25% to about 50% by weight of water may comprise about 5% to about 30%, about 10% to about 30%, about 15% to about 30%, about 20% to about 30%, about 25% to about 30%, about 5% to about 25%, about 10% to about 25%, about 15% to about 25%, about 20% to about 25%, about 5% to about 20%, about 10% to about 20%, about 15% to about 20%, about 3% to about 15%, about 4% to about 15%, about 5% to about 15%, about 6% to about 15%, 7% to about 15%, about 8% to about 15%, about 9% to about 15%, about 10% to about 15%, about 11% to about 15%, about 12% to about 15%, about 13% to about 15%, about 14% to about 15%, about 3% to about 14%, about 4% to about 14%, about 5% to about 14%, about 6% to about 14%, about 7% to about 14%, about 8% to about 14%, about 9% to about 14%, about 10% to about 14%, about 11% to about 14%, about 12% to about 14%, about 13% to about 14%, about 3% to about 13%, about 4% to about 13%, about 5% to about 13%, about 6% to about 13%, about 7% to about 13%, about 8% to about 13%, about 9% to about 13%, about 3% to about 12%, about 4% to about 12%, about 5% to about 12%, about 6% to about 12%, about 7% to about 12%, about 8% to about 12%, about 9% to about 12%, about 10% to about 12%, about 11% to about 12%, about 3% to about 11%, about 4% to about 11%, about 5% to about 11%, about 6% to about 11%, about 7% to about 11%, about 8% to about 11%, about 9% to about 11%, about 10% to about 11%, about 3% to about 10%, about 4% to about 10%, about 5% to about 10%, about 6% to about 10%, about 7% to about 10%, about 8% to about 10%, about 9% to about 10%, about 3% to about 9%, about 4% to about 9%, about 5% to about 9%, about 6% to about 9%, about 7% to about 9%, about 8% to about 9%, about 3% to about 8%, about 4% to about 8%, about 5% to about 8%, about 6% to about 8%, about 7% to about 8%, about 3% to about 7%, about 4% to about 7%, about 5% to about 7%, about 6% to about 7%, about 3% to about 6%, about 4% to about 6%, about 5% to about 6%, about 3% to about 5%, about 4% to about 5%, or about 3% to about 4% by weight of penetration enhancer such as caprylic/capric glycerides or PEGylated caprylic/capric glycerides, preferably PEG-6 caprylic/capric glycerides.

Compositions comprising more than about 40% by weight of synephrine may comprise no penetration enhancer. Preferably, such formulations with no penetration enhancer comprise at least 40%, 45%, 50%, 55%, 60%, 65% or 70% w/w synephrine.

Additives

The synephrine compositions of the present disclosure also may include additives. One additive that is present in some embodiments is glycolic acid, which is available as an aqueous solution, such as 70% glycolic acid by weight in aqueous solution. The following compositions comprise glycolic acid in which the weight percentages given are the weight percentage of 70% glycolic acid in aqueous solution based on the weight of the total composition. Other concentrations of glycolic acid solutions may be used within the scope of the invention. One composition of the present disclosure comprises between about 10% to about 60% by weight of synephrine, between about 10% and about 60% by weight water, between about 5% and about 30% by weight PEGylated or nonPEGylated caprylic/capric glyceride, and between about 5% to about 25% glycolic acid solution. In other embodiments, synephrine is present at about 40% to 50%, water is present at about 30% to 50%, PEGylated caprylic/capric glyceride is present at about 5% to 20%, and glycolic acid solution is present at between about 5% to about 10%. In other embodiments, synephrine is present at about 30% to 50%, water at about 20% to 40%, PEGylated caprylic/capric glyceride at about 5% to 20%, and glycolic acid solution is present at between about 5% to about 25%. In a further embodiment, synephrine is present at about 20% to 30%, water is present at about 10% to about 40%, PEGylated caprylic/capric glyceride is present at about 5% to 20%, and glycolic acid solution is present at about 5% to about 20%.

The composition may comprise as a further additive a diol, such as a C₃ to C₈ alkyl diol, including without limitation propanediol, such as 1,3 propanediol or 1,2-propanediol, which diol preferably functions as a preservative. The diol, preferably propanediol, is present in the composition at about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3.5%, about 0.05% to about 3%, about 0.05% to about 2.5%, about 0.05% to about 2%, about 0.05% to about 1%, about 0.8% to about 10%, about 0.8% to about 9%, about 0.8% to about 8%, about 0.8% to about 7%, about 0.8% to about 6%, about 0.8% to about 5%, about 0.8% to about 4%, about 0.8% to about 3.5%, about 0.8% to about 3%, about 0.8% to about 2.5%, about 0.8% to about 2%, about 0.8% to about 1%, about 1% to about 10%, about 1% to about 9%, about 1% to about 8%, about 1% to about 7%, about 1% to about 6%, about 1% to about 5%, about 1% to about 4%, about 1% to about 3.5%, about 1% to about 3%, about 1% to about 2.5%, about 1% to about 2%, about 1.2% to about 10%, about 1.2% to about 9%, about 1.2% to about 8%, about 1.2% to about 7%, about 1.2% to about 6%, about 1.2% to about 5%, about 1.2% to about 4%, about 1.2% to about 3.5%, about 1.2% to about 3%, about 1.2% to about 2.5%, about 1.2% to about 2%, about 1.5% to about 10%, about 1.5% to about 9%, about 1.5% to about 8%, about 1.5% to about 7%, about 1.5% to about 6%, about 1.5% to about 5%, about 1.5% to about 4%, about 1.5% to about 3.5%, about 1.5% to about 3%, about 1.5% to about 2.5%, about 1.5% to about 2%, about 1.7% to about 10%, about 1.7% to about 9%, about 1.7% to about 8%, about 1.7% to about 7%, about 1.7% to about 6%, about 1.7% to about 5%, about 1.7% to about 4%, about 1.7% to about 3.5%, about 1.7% to about 3%, about 1.7% to about 2.5%, about 1.7% to about 2%, about 1.8% to about 10%, about 1.8% to about 9%, about 1.8% to about 8%, about 1.8% to about 7%, about 1.8% to about 6%, about 1.8% to about 5%, about 1.8% to about 4%, about 1.8% to about 3.5%, about 1.8% to about 3%, about 1.8% to about 2.5%, about 1.8% to about 2%, about 2% to about 10%, about 2% to about 9%, about 2% to about 8%, about 2% to about 7%, about 2% to about 6%, about 2% to about 5%, about 2% to about 4%, about 2% to about 3.5%, about 2% to about 3%, about 2% to about 2.5%, about 2% to about 2%, about 2% to about 1%, about 2.1% to about 10%, about 2.1% to about 9%, about 2.1% to about 8%, about 2.1% to about 7%, about 2.1% to about 6%, about 2.1% to about 5%, about 2.1% to about 4%, about 2.1% to about 3.5%, about 2.1% to about 3%, about 2.1% to about 2.5%, about 2.2% to about 10%, about 2.2% to about 9%, about 2.2% to about 8%, about 2.2% to about 7%, about 2.2% to about 6%, about 2.2% to about 5%, about 2.2% to about 4%, about 2.2% to about 3.5%, about 2.2% to about 3%, about 2.2% to about 2.5%, about 2.3% to about 10%, about 2.3% to about 9%, about 2.3% to about 8%, about 2.3% to about 7%, about 2.3% to about 6%, about 2.3% to about 5%, about 2.3% to about 4%, about 2.3% to about 3.5%, about 2.3% to about 3%, about 2.3% to about 2.5%, about 2.4% to about 10%, about 2.4% to about 9%, about 2.4% to about 8%, about 2.4% to about 7%, about 2.4% to about 6%, about 2.4% to about 5%, about 2.4% to about 4%, about 2.4% to about 3.5%, about 2.4% to about 3%, about 2.4% to about 2.5%, about 2.5% to about 10%, about 2.5% to about 9%, about 2.5% to about 8%, about 2.5% to about 7%, about 2.5% to about 6%, about 2.5% to about 5%, about 2.5% to about 4%, about 2.5% to about 3.5%, about 2.5% to about 3%, about 2.6% to about 10%, about 2.6% to about 9%, about 2.6% to about 8%, about 2.6% to about 7%, about 2.6% to about 6%, about 2.6% to about 5%, about 2.6% to about 4%, about 2.6% to about 3.5%, about 2.6% to about 3%, about 2.7% to about 10%, about 2.7% to about 9%, about 2.7% to about 8%, about 2.7% to about 7%, about 2.7% to about 6%, about 2.7% to about 5%, about 2.7% to about 4%, about 2.7% to about 3.5%, about 2.7% to about 3%, about 2.8% to about 10%, about 2.8% to about 9%, about 2.8% to about 8%, about 2.8% to about 7%, about 2.8% to about 6%, about 2.8% to about 5%, about 2.8% to about 4%, about 2.8% to about 3.5%, about 2.8% to about 3%, about 2.9% to about 10%, about 2.9% to about 9%, about 2.9% to about 8%, about 2.9% to about 7%, about 2.9% to about 6%, about 2.9% to about 5%, about 2.9% to about 4%, about 2.9% to about 3.5%, about 2.9% to about 3%, about 3% to about 10%, about 3% to about 9%, about 3% to about 8%, about 3% to about 7%, about 3% to about 6%, about 3% to about 5%, about 3% to about 4%, about 3% to about 3.5%, about 3% to about 3%, about 3% to about 2.5%, about 3% to about 2%, about 3% to about 1%, about 3.1% to about 10%, about 3.1% to about 9%, about 3.1% to about 8%, about 3.1% to about 7%, about 3.1% to about 6%, about 3.1% to about 5%, about 3.1% to about 4%, about 3.1% to about 3.5%, about 3.2% to about 10%, about 3.2% to about 9%, about 3.2% to about 8%, about 3.2% to about 7%, about 3.2% to about 6%, about 3.2% to about 5%, about 3.2% to about 4%, about 3.2% to about 3.5%, about 3.3% to about 10%, about 3.3% to about 9%, about 3.3% to about 8%, about 3.3% to about 7%, about 3.3% to about 6%, about 3.3% to about 5%, about 3.3% to about 4%, about 3.3% to about 3.5%, about 3.4% to about 10%, about 3.4% to about 9%, about 3.4% to about 8%, about 3.4% to about 7%, about 3.4% to about 6%, about 3.4% to about 5%, about 3.4% to about 4%, about 3.4% to about 3.5%, about 3.5% to about 10%, about 3.5% to about 9%, about 3.5% to about 8%, about 3.5% to about 7%, about 3.5% to about 6%, about 3.5% to about 5%, about 3.5% to about 4%, about 3.5% to about 3.9%, about 3.5% to about 3.8%, about 3.5% to about 3.7%, about 3.5% to about 3.6%, about 3.6% to about 10%, about 3.6% to about 9%, about 3.6% to about 8%, about 3.6% to about 7%, about 3.6% to about 6%, about 3.6% to about 5%, about 3.6% to about 4%, about 3.6% to about 3.9%, about 3.6% to about 3.8%, about 3.6% to about 3.7%, about 3.7% to about 10%, about 3.7% to about 9%, about 3.7% to about 8%, about 3.7% to about 7%, about 3.7% to about 6%, about 3.7% to about 5%, about 3.7% to about 4%, about 3.7% to about 3.9%, about 3.7% to about 3.8%, about 3.8% to about 10%, about 3.8% to about 9%, about 3.8% to about 8%, about 3.8% to about 7%, about 3.8% to about 6%, about 3.8% to about 5%, about 3.8% to about 4%, about 3.8% to about 3.9%, about 3.9% to about 10%, about 3.9% to about 9%, about 3.9% to about 8%, about 3.9% to about 7%, about 3.9% to about 6%, about 3.9% to about 5%, about 3.9% to about 4% about 4% to about 10%, about 4% to about 9%, about 4% to about 8%, about 4% to about 7%, about 4% to about 6%, about 4% to about 5%, about 4% to about 4.5%, about 4.5% to about 10%, about 4.5% to about 9%, about 4.5% to about 8%, about 4.5% to about 7%, about 4.5% to about 6%, about 4.5% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% by weight of the total composition.

The composition may also comprise phenoxyethanol ethylhexylglycerin, (such as the product EUXYL PE 9010 sold by Schuelke Inc.), which acts as a preservative, in a weight percent as percentage of the total weight of the aqueous composition of about 0.05% to about 5%, about 0.05% to about 4%, about 0.05% to about 3%, about 0.05% to about 2%, about 0.05% to about 1.9%, about 0.05% to about 1.8%, about 0.05% to about 1.7%, about 0.05% to about 1.6%, about 0.05% to about 1.5%, about 0.05% to about 1.4%, about 0.05% to about 1.3%, about 0.05% to about 1.2%, about 0.05% to about 1.1%, about 0.05% to about 1%, about 0.05% to about 0.9%, about 0.05% to about 0.8%, about 0.05% to about 0.7%, about 0.05% to about 0.6%, about 0.05% to about 0.5%, about 0.1% to about 5%, about 0.1% to about 4%, about 0.1% to about 3%, about 0.1% to about 2%, about 0.1% to about 1.9%, about 0.1% to about 1.8%, about 0.1% to about 1.7%, about 0.1% to about 1.6%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.2% to about 5%, about 0.2% to about 4%, about 0.2% to about 3%, about 0.2% to about 2%, about 0.2% to about 1.9%, about 0.2% to about 1.8%, about 0.2% to about 1.7%, about 0.2% to about 1.6%, about 0.2% to about 1.5%, about 0.2% to about 1.4%, about 0.2% to about 1.3%, about 0.2% to about 1.2%, about 0.2% to about 1.1%, about 0.2% to about 1%, about 0.2% to about 0.9%, about 0.2% to about 0.8%, about 0.2% to about 0.7%, about 0.2% to about 0.6%, about 0.2% to about 0.5%, about 0.3% to about 5%, about 0.3% to about 4%, about 0.3% to about 3%, about 0.3% to about 2%, about 0.3% to about 1.9%, about 0.3% to about 1.8%, about 0.3% to about 1.7%, about 0.3% to about 1.6%, about 0.3% to about 1.5%, about 0.3% to about 1.4%, about 0.3% to about 1.3%, about 0.3% to about 1.2%, about 0.3% to about 1.1%, about 0.3% to about 1%, about 0.3% to about 0.9%, about 0.3% to about 0.8%, about 0.3% to about 0.7%, about 0.3% to about 0.6%, about 0.3% to about 0.5%, about 0.4% to about 5%, about 0.4% to about 4%, about 0.4% to about 3%, about 0.4% to about 2%, about 0.4% to about 1.9%, about 0.4% to about 1.8%, about 0.4% to about 1.7%, about 0.4% to about 1.6%, about 0.4% to about 1.5%, about 0.4% to about 1.4%, about 0.4% to about 1.3%, about 0.4% to about 1.2%, about 0.4% to about 1.1%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 5%, about 0.5% to about 4%, about 0.5% to about 3%, about 0.5% to about 2%, about 0.5% to about 1.9%, about 0.5% to about 1.8%, about 0.5% to about 1.7%, about 0.5% to about 1.6%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, about 0.5% to about 0.6%, about 0.6% to about 5%, about 0.6% to about 4%, about 0.6% to about 3%, about 0.6% to about 2%, about 0.6% to about 1.9%, about 0.6% to about 1.8%, about 0.6% to about 1.7%, about 0.6% to about 1.6%, about 0.6% to about 1.5%, about 0.6% to about 1.4%, about 0.6% to about 1.3%, about 0.6% to about 1.2%, about 0.6% to about 1.1%, about 0.6% to about 1%, about 0.6% to about 0.9%, about 0.6% to about 0.8%, about 0.6% to about 0.7%, about 0.7% to about 5%, about 0.7% to about 4%, about 0.7% to about 3%, about 0.7% to about 2%, about 0.7% to about 1.9%, about 0.7% to about 1.8%, about 0.7% to about 1.7%, about 0.7% to about 1.6%, about 0.7% to about 1.5%, about 0.7% to about 1.4%, about 0.7% to about 1.3%, about 0.7% to about 1.2%, about 0.7% to about 1.1%, about 0.7% to about 1%, about 0.7% to about 0.9%, about 0.7% to about 0.8%, about 0.8% to about 5%, about 0.8% to about 4%, about 0.8% to about 3%, about 0.8% to about 2%, about 0.8% to about 1.9%, about 0.8% to about 1.8%, about 0.8% to about 1.7%, about 0.8% to about 1.6%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 5%, about 0.9% to about 4%, about 0.9% to about 3%, about 0.9% to about 2%, about 0.9% to about 1.9%, about 0.9% to about 1.8%, about 0.9% to about 1.7%, about 0.9% to about 1.6%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 5%, about 1% to about 4%, about 1% to about 3%, about 1% to about 2%, about 1% to about 1.9%, about 1% to about 1.8%, about 1% to about 1.7%, about 1% to about 1.6%, about 1% to about 1.5%, about 1% to about 1.4%, about 1% to about 1.3%, about 1% to about 1.2%, about 1% to about 1.1%, about 1.1% to about 5%, about 1.1% to about 4%, about 1.1% to about 3%, about 1.1% to about 2%, about 1.1% to about 1.9%, about 1.1% to about 1.8%, about 1.1% to about 1.7%, about 1.1% to about 1.6%, about 1.1% to about 1.5%, about 1.1% to about 1.4%, about 1.1% to about 1.3%, about 1.1% to about 1.2%, about 1.2% to about 5%, about 1.2% to about 4%, about 1.2% to about 3%, about 1.2% to about 2%, about 1.2% to about 1.9%, about 1.2% to about 1.8%, about 1.2% to about 1.7%, about 1.2% to about 1.6%, about 1.2% to about 1.5%, about 1.2% to about 1.4%, about 1.2% to about 1.3%, about 1.3% to about 5%, about 1.3% to about 4%, about 1.3% to about 3%, about 1.3% to about 2%, about 1.3% to about 1.9%, about 1.3% to about 1.8%, about 1.3% to about 1.7%, about 1.3% to about 1.6%, about 1.3% to about 1.5%, about 1.3% to about 1.4%, about 1.4% to about 5%, about 1.4% to about 4%, about 1.4% to about 3%, about 1.4% to about 2%, about 1.4% to about 1.9%, about 1.4% to about 1.8%, about 1.4% to about 1.7%, about 1.4% to about 1.6%, about 1.4% to about 1.5%, about 1.5% to about 5%, about 1.5% to about 4%, about 1.5% to about 3%, about 1.5% to about 2%, about 1.5% to about 1.9%, about 1.5% to about 1.8%, about 1.5% to about 1.7%, or about 1.5% to about 1.6%.

In some embodiments, the composition further comprises at least one fragrance. Suitable fragrances are known to those of ordinary skill in the art. Additional additives may be needed to aid solubilizing the fragrance in the composition, as could readily be determined by one of ordinary skill in cosmetic or pharmaceutical formulations.

The composition may comprise an acid such as hydrochloric acid or a base such as NaCl to aid solubilizing, especially in synephrine formulations including glycolic acid.

The composition may comprise one or more antioxidants and preservatives in place of or in addition to the additives discussed above that act as preservatives, such as without limitation tetrasodium EDTA, sodium metabisulfite, and butylated hydroxytoluene (BHT). The amount of each antioxidant or preservative in the present compositions based on the total weight of the composition is about 0.01% to about 5%, about 0.01% to about 4%, about 0.01% to about 3%, about 0.01% to about 2%, about 0.01% to about 1%, about 0.01% to about 0.9%, about 0.01% to about 0.8%, about 0.01% to about 0.7%, about 0.01% to about 0.6%, about 0.01% to about 0.5%, about 0.01% to about 0.4%, about 0.01% to about 0.3%, about 0.01% to about 0.2%, about 0.01% to about 0.1%, about 0.03% to about 5%, about 0.03% to about 4%, about 0.03% to about 3%, about 0.03% to about 2%, about 0.03% to about 1%, about 0.03% to about 0.9%, about 0.03% to about 0.8%, about 0.03% to about 0.7%, about 0.03% to about 0.6%, about 0.03% to about 0.5%, about 0.03% to about 0.4%, about 0.03% to about 0.3%, about 0.03% to about 0.2%, about 0.03% to about 0.1%, about 0.04% to about 5%, about 0.04% to about 4%, about 0.04% to about 3%, about 0.04% to about 2%, about 0.04% to about 1%, about 0.04% to about 0.9%, about 0.04% to about 0.8%, about 0.04% to about 0.7%, about 0.04% to about 0.6%, about 0.04% to about 0.5%, about 0.04% to about 0.4%, about 0.04% to about 0.3%, about 0.04% to about 0.2%, about 0.04% to about 0.1%, about 0.05% to about 5%, about 0.05% to about 4%, about 0.05% to about 3%, about 0.05% to about 2%, about 0.05% to about 1%, about 0.05% to about 0.9%, about 0.05% to about 0.8%, about 0.05% to about 0.7%, about 0.05% to about 0.6%, about 0.05% to about 0.5%, about 0.05% to about 0.4%, about 0.05% to about 0.3%, about 0.05% to about 0.2%, about 0.05% to about 0.1%, about 0.06% to about 5%, about 0.06% to about 4%, about 0.06% to about 3%, about 0.06% to about 2%, about 0.06% to about 1%, about 0.06% to about 0.9%, about 0.06% to about 0.8%, about 0.06% to about 0.7%, about 0.06% to about 0.6%, about 0.06% to about 0.5%, about 0.06% to about 0.4%, about 0.06% to about 0.3%, about 0.06% to about 0.2%, about 0.06% to about 0.1%, about 0.07% to about 5%, about 0.07% to about 4%, about 0.07% to about 3%, about 0.07% to about 2%, about 0.07% to about 1%, about 0.07% to about 0.9%, about 0.07% to about 0.8%, about 0.07% to about 0.7%, about 0.07% to about 0.6%, about 0.07% to about 0.5%, about 0.07% to about 0.4%, about 0.07% to about 0.3%, about 0.07% to about 0.2%, about 0.07% to about 0.1%, about 0.08% to about 5%, about 0.08% to about 4%, about 0.08% to about 3%, about 0.08% to about 2%, about 0.08% to about 1%, about 0.08% to about 0.9%, about 0.08% to about 0.8%, about 0.08% to about 0.7%, about 0.08% to about 0.6%, about 0.08% to about 0.5%, about 0.08% to about 0.4%, about 0.08% to about 0.3%, about 0.08% to about 0.2%, about 0.08% to about 0.1%, about 0.09% to about 5%, about 0.09% to about 4%, about 0.09% to about 3%, about 0.09% to about 2%, about 0.09% to about 1%, about 0.09% to about 0.9%, about 0.09% to about 0.8%, about 0.09% to about 0.7%, about 0.09% to about 0.6%, about 0.09% to about 0.5%, about 0.09% to about 0.4%, about 0.09% to about 0.3%, about 0.09% to about 0.2%, about 0.09% to about 0.1%, about 0.1% to about 5%, about 0.1% to about 4%, about 0.1% to about 3%, about 0.1% to about 2%, about 0.1% to about 1.5%, about 0.1% to about 1%, about 0.1% to about 0.9%, about 0.8% to about 0.7%, about 0.1% to about 0.6%, about 0.1% to about 0.5%, about 0.1% to about 0.4%, about 0.1% to about 0.3%, about 0.1% to about 0.2%, about 0.2% to about 5%, about 0.2% to about 4%, about 0.2% to about 3%, about 0.2% to about 2%, about 0.2% to about 1.5%, about 0.2% to about 1%, about 0.2% to about 0.9%, about 0.8% to about 0.7%, about 0.2% to about 0.6%, about 0.2% to about 0.5%, about 0.2% to about 0.4%, about 0.2% to about 0.3%, about 0.3% to about 5%, about 0.3% to about 4%, about 0.3% to about 3%, about 0.3% to about 2%, about 0.3% to about 1.5%, about 0.3% to about 1%, about 0.3% to about 0.9%, about 0.8% to about 0.7%, about 0.3% to about 0.6%, about 0.3% to about 0.5%, about 0.3% to about 0.4%, about 0.4% to about 5%, about 0.4% to about 4%, about 0.4% to about 3%, about 0.4% to about 2%, about 0.4% to about 1.5%, about 0.4% to about 1%, about 0.4% to about 0.9%, about 0.8% to about 0.7%, about 0.4% to about 0.6%, or about 0.4% to about 0.5%.

The composition may comprise one or more moisturizers (also known as humectants), such as without limitation panthenol. A moisturizer may be present in the composition at a weight percentage based on the total weight of the composition of about 0.01% to about 5%, about 0.01% to about 4%, about 0.01% to about 3%, about 0.01% to about 2%, about 0.01% to about 1%, about 0.01% to about 0.9%, about 0.01% to about 0.8%, about 0.01% to about 0.7%, about 0.01% to about 0.6%, about 0.01% to about 0.5%, about 0.01% to about 0.4%, about 0.01% to about 0.3%, about 0.01% to about 0.2%, about 0.01% to about 0.1%, about 0.03% to about 5%, about 0.03% to about 4%, about 0.03% to about 3%, about 0.03% to about 2%, about 0.03% to about 1%, about 0.03% to about 0.9%, about 0.03% to about 0.8%, about 0.03% to about 0.7%, about 0.03% to about 0.6%, about 0.03% to about 0.5%, about 0.03% to about 0.4%, about 0.03% to about 0.3%, about 0.03% to about 0.2%, about 0.03% to about 0.1%, about 0.04% to about 5%, about 0.04% to about 4%, about 0.04% to about 3%, about 0.04% to about 2%, about 0.04% to about 1%, about 0.04% to about 0.9%, about 0.04% to about 0.8%, about 0.04% to about 0.7%, about 0.04% to about 0.6%, about 0.04% to about 0.5%, about 0.04% to about 0.4%, about 0.04% to about 0.3%, about 0.04% to about 0.2%, about 0.04% to about 0.1%, about 0.05% to about 5%, about 0.05% to about 4%, about 0.05% to about 3%, about 0.05% to about 2%, about 0.05% to about 1%, about 0.05% to about 0.9%, about 0.05% to about 0.8%, about 0.05% to about 0.7%, about 0.05% to about 0.6%, about 0.05% to about 0.5%, about 0.05% to about 0.4%, about 0.05% to about 0.3%, about 0.05% to about 0.2%, about 0.05% to about 0.1%, about 0.06% to about 5%, about 0.06% to about 4%, about 0.06% to about 3%, about 0.06% to about 2%, about 0.06% to about 1%, about 0.06% to about 0.9%, about 0.06% to about 0.8%, about 0.06% to about 0.7%, about 0.06% to about 0.6%, about 0.06% to about 0.5%, about 0.06% to about 0.4%, about 0.06% to about 0.3%, about 0.06% to about 0.2%, about 0.06% to about 0.1%, about 0.07% to about 5%, about 0.07% to about 4%, about 0.07% to about 3%, about 0.07% to about 2%, about 0.07% to about 1%, about 0.07% to about 0.9%, about 0.07% to about 0.8%, about 0.07% to about 0.7%, about 0.07% to about 0.6%, about 0.07% to about 0.5%, about 0.07% to about 0.4%, about 0.07% to about 0.3%, about 0.07% to about 0.2%, about 0.07% to about 0.1%, about 0.08% to about 5%, about 0.08% to about 4%, about 0.08% to about 3%, about 0.08% to about 2%, about 0.08% to about 1%, about 0.08% to about 0.9%, about 0.08% to about 0.8%, about 0.08% to about 0.7%, about 0.08% to about 0.6%, about 0.08% to about 0.5%, about 0.08% to about 0.4%, about 0.08% to about 0.3%, about 0.08% to about 0.2%, about 0.08% to about 0.1%, about 0.09% to about 5%, about 0.09% to about 4%, about 0.09% to about 3%, about 0.09% to about 2%, about 0.09% to about 1%, about 0.09% to about 0.9%, about 0.09% to about 0.8%, about 0.09% to about 0.7%, about 0.09% to about 0.6%, about 0.09% to about 0.5%, about 0.09% to about 0.4%, about 0.09% to about 0.3%, about 0.09% to about 0.2%, about 0.09% to about 0.1%, about 0.1% to about 5%, about 0.1% to about 4%, about 0.1% to about 3%, about 0.1% to about 2%, about 0.1% to about 1.5%, about 0.1% to about 1%, about 0.1% to about 0.9%, about 0.8% to about 0.7%, about 0.1% to about 0.6%, about 0.1% to about 0.5%, about 0.1% to about 0.4%, about 0.1% to about 0.3%, about 0.1% to about 0.2%, about 0.2% to about 5%, about 0.2% to about 4%, about 0.2% to about 3%, about 0.2% to about 2%, about 0.2% to about 1.5%, about 0.2% to about 1%, about 0.2% to about 0.9%, about 0.8% to about 0.7%, about 0.2% to about 0.6%, about 0.2% to about 0.5%, about 0.2% to about 0.4%, about 0.2% to about 0.3%, about 0.3% to about 5%, about 0.3% to about 4%, about 0.3% to about 3%, about 0.3% to about 2%, about 0.3% to about 1.5%, about 0.3% to about 1%, about 0.3% to about 0.9%, about 0.8% to about 0.7%, about 0.3% to about 0.6%, about 0.3% to about 0.5%, about 0.3% to about 0.4%, about 0.4% to about 5%, about 0.4% to about 4%, about 0.4% to about 3%, about 0.4% to about 2%, about 0.4% to about 1.5%, about 0.4% to about 1%, about 0.4% to about 0.9%, about 0.8% to about 0.7%, about 0.4% to about 0.6%, or about 0.4% to about 0.5%.

The composition further may comprise niacinamide, which is a known additive in hair care products. Niacinamide may be present in the composition at a weight percentage based on the total weight of the composition of about 0.01% to about 5%, about 0.01% to about 4%, about 0.01% to about 3%, about 0.01% to about 2%, about 0.01% to about 1%, about 0.01% to about 0.9%, about 0.01% to about 0.8%, about 0.01% to about 0.7%, about 0.01% to about 0.6%, about 0.01% to about 0.5%, about 0.01% to about 0.4%, about 0.01% to about 0.3%, about 0.01% to about 0.2%, about 0.01% to about 0.1%, about 0.03% to about 5%, about 0.03% to about 4%, about 0.03% to about 3%, about 0.03% to about 2%, about 0.03% to about 1%, about 0.03% to about 0.9%, about 0.03% to about 0.8%, about 0.03% to about 0.7%, about 0.03% to about 0.6%, about 0.03% to about 0.5%, about 0.03% to about 0.4%, about 0.03% to about 0.3%, about 0.03% to about 0.2%, about 0.03% to about 0.1%, about 0.04% to about 5%, about 0.04% to about 4%, about 0.04% to about 3%, about 0.04% to about 2%, about 0.04% to about 1%, about 0.04% to about 0.9%, about 0.04% to about 0.8%, about 0.04% to about 0.7%, about 0.04% to about 0.6%, about 0.04% to about 0.5%, about 0.04% to about 0.4%, about 0.04% to about 0.3%, about 0.04% to about 0.2%, about 0.04% to about 0.1%, about 0.05% to about 5%, about 0.05% to about 4%, about 0.05% to about 3%, about 0.05% to about 2%, about 0.05% to about 1%, about 0.05% to about 0.9%, about 0.05% to about 0.8%, about 0.05% to about 0.7%, about 0.05% to about 0.6%, about 0.05% to about 0.5%, about 0.05% to about 0.4%, about 0.05% to about 0.3%, about 0.05% to about 0.2%, about 0.05% to about 0.1%, about 0.06% to about 5%, about 0.06% to about 4%, about 0.06% to about 3%, about 0.06% to about 2%, about 0.06% to about 1%, about 0.06% to about 0.9%, about 0.06% to about 0.8%, about 0.06% to about 0.7%, about 0.06% to about 0.6%, about 0.06% to about 0.5%, about 0.06% to about 0.4%, about 0.06% to about 0.3%, about 0.06% to about 0.2%, about 0.06% to about 0.1%, about 0.07% to about 5%, about 0.07% to about 4%, about 0.07% to about 3%, about 0.07% to about 2%, about 0.07% to about 1%, about 0.07% to about 0.9%, about 0.07% to about 0.8%, about 0.07% to about 0.7%, about 0.07% to about 0.6%, about 0.07% to about 0.5%, about 0.07% to about 0.4%, about 0.07% to about 0.3%, about 0.07% to about 0.2%, about 0.07% to about 0.1%, about 0.08% to about 5%, about 0.08% to about 4%, about 0.08% to about 3%, about 0.08% to about 2%, about 0.08% to about 1%, about 0.08% to about 0.9%, about 0.08% to about 0.8%, about 0.08% to about 0.7%, about 0.08% to about 0.6%, about 0.08% to about 0.5%, about 0.08% to about 0.4%, about 0.08% to about 0.3%, about 0.08% to about 0.2%, about 0.08% to about 0.1%, about 0.09% to about 5%, about 0.09% to about 4%, about 0.09% to about 3%, about 0.09% to about 2%, about 0.09% to about 1%, about 0.09% to about 0.9%, about 0.09% to about 0.8%, about 0.09% to about 0.7%, about 0.09% to about 0.6%, about 0.09% to about 0.5%, about 0.09% to about 0.4%, about 0.09% to about 0.3%, about 0.09% to about 0.2%, about 0.09% to about 0.1%, about 0.1% to about 5%, about 0.1% to about 4%, about 0.1% to about 3%, about 0.1% to about 2%, about 0.1% to about 1.5%, about 0.1% to about 1%, about 0.1% to about 0.9%, about 0.8% to about 0.7%, about 0.1% to about 0.6%, about 0.1% to about 0.5%, about 0.1% to about 0.4%, about 0.1% to about 0.3%, about 0.1% to about 0.2%, about 0.2% to about 5%, about 0.2% to about 4%, about 0.2% to about 3%, about 0.2% to about 2%, about 0.2% to about 1.5%, about 0.2% to about 1%, about 0.2% to about 0.9%, about 0.8% to about 0.7%, about 0.2% to about 0.6%, about 0.2% to about 0.5%, about 0.2% to about 0.4%, or about 0.2% to about 0.3%.

Surfactants and emulsifiers may also be present in the compositions, including without limitation polysorbate 80 (also known as TWEEN 80). A surfactant or emulsifier may function to solubilize additives, such as the fragrance. One or more surfactants or emulsifiers may be present in the composition at a weight percentage for each emulsifier or surfactant based on the total weight of the composition of about 0.01% to about 5%, about 0.01% to about 4%, about 0.01% to about 3%, about 0.01% to about 2%, about 0.01% to about 1%, about 0.01% to about 0.9%, about 0.01% to about 0.8%, about 0.01% to about 0.7%, about 0.01% to about 0.6%, about 0.01% to about 0.5%, about 0.01% to about 0.4%, about 0.01% to about 0.3%, about 0.01% to about 0.2%, about 0.01% to about 0.1%, about 0.03% to about 5%, about 0.03% to about 4%, about 0.03% to about 3%, about 0.03% to about 2%, about 0.03% to about 1%, about 0.03% to about 0.9%, about 0.03% to about 0.8%, about 0.03% to about 0.7%, about 0.03% to about 0.6%, about 0.03% to about 0.5%, about 0.03% to about 0.4%, about 0.03% to about 0.3%, about 0.03% to about 0.2%, about 0.03% to about 0.1%, about 0.04% to about 5%, about 0.04% to about 4%, about 0.04% to about 3%, about 0.04% to about 2%, about 0.04% to about 1%, about 0.04% to about 0.9%, about 0.04% to about 0.8%, about 0.04% to about 0.7%, about 0.04% to about 0.6%, about 0.04% to about 0.5%, about 0.04% to about 0.4%, about 0.04% to about 0.3%, about 0.04% to about 0.2%, about 0.04% to about 0.1%, about 0.05% to about 5%, about 0.05% to about 4%, about 0.05% to about 3%, about 0.05% to about 2%, about 0.05% to about 1%, about 0.05% to about 0.9%, about 0.05% to about 0.8%, about 0.05% to about 0.7%, about 0.05% to about 0.6%, about 0.05% to about 0.5%, about 0.05% to about 0.4%, about 0.05% to about 0.3%, about 0.05% to about 0.2%, about 0.05% to about 0.1%, about 0.06% to about 5%, about 0.06% to about 4%, about 0.06% to about 3%, about 0.06% to about 2%, about 0.06% to about 1%, about 0.06% to about 0.9%, about 0.06% to about 0.8%, about 0.06% to about 0.7%, about 0.06% to about 0.6%, about 0.06% to about 0.5%, about 0.06% to about 0.4%, about 0.06% to about 0.3%, about 0.06% to about 0.2%, about 0.06% to about 0.1%, about 0.07% to about 5%, about 0.07% to about 4%, about 0.07% to about 3%, about 0.07% to about 2%, about 0.07% to about 1%, about 0.07% to about 0.9%, about 0.07% to about 0.8%, about 0.07% to about 0.7%, about 0.07% to about 0.6%, about 0.07% to about 0.5%, about 0.07% to about 0.4%, about 0.07% to about 0.3%, about 0.07% to about 0.2%, about 0.07% to about 0.1%, about 0.08% to about 5%, about 0.08% to about 4%, about 0.08% to about 3%, about 0.08% to about 2%, about 0.08% to about 1%, about 0.08% to about 0.9%, about 0.08% to about 0.8%, about 0.08% to about 0.7%, about 0.08% to about 0.6%, about 0.08% to about 0.5%, about 0.08% to about 0.4%, about 0.08% to about 0.3%, about 0.08% to about 0.2%, about 0.08% to about 0.1%, about 0.09% to about 5%, about 0.09% to about 4%, about 0.09% to about 3%, about 0.09% to about 2%, about 0.09% to about 1%, about 0.09% to about 0.9%, about 0.09% to about 0.8%, about 0.09% to about 0.7%, about 0.09% to about 0.6%, about 0.09% to about 0.5%, about 0.09% to about 0.4%, about 0.09% to about 0.3%, about 0.09% to about 0.2%, about 0.09% to about 0.1%, about 0.1% to about 5%, about 0.1% to about 4%, about 0.1% to about 3%, about 0.1% to about 2%, about 0.1% to about 1.5%, about 0.1% to about 1%, about 0.1% to about 0.9%, about 0.8% to about 0.7%, about 0.1% to about 0.6%, about 0.1% to about 0.5%, about 0.1% to about 0.4%, about 0.1% to about 0.3%, about 0.1% to about 0.2%, about 0.2% to about 5%, about 0.2% to about 4%, about 0.2% to about 3%, about 0.2% to about 2%, about 0.2% to about 1.5%, about 0.2% to about 1%, about 0.2% to about 0.9%, about 0.8% to about 0.7%, about 0.2% to about 0.6%, about 0.2% to about 0.5%, about 0.2% to about 0.4%, or about 0.2% to about 0.3%.

Exemplary Compositions

In some embodiments, the composition comprises water, synephrine, PEG-6 caprylic/capric glycerides, propanediol, phenoxyethanol, sodium metabisulfite, panthenol, niacinamide, butylated hydroxytoluene, polysorbate 80, phenoxyethanol and tetrasodium EDTA (ethylenediamenetraacetic acid tetrasodium salt). In one or more embodiments, the composition further comprises a fragrance.

In some embodiments, the composition consists essentially of water, synephrine, PEG-6 caprylic/capric glycerides, propanediol, phenoxyethanol, sodium metabisulfite, panthenol, niacinamide, butylated hydroxytoluene, polysorbate 80, phenoxyethanol and tetrasodium EDTA (ethylenediamenetraacetic acid tetrasodium salt) and fragrance.

In some embodiments, the composition consists of water, synephrine, PEG-6 caprylic/capric glycerides, propanediol, phenoxyethanol, sodium metabisulfite, panthenol, niacinamide, butylated hydroxytoluene, polysorbate 80, phenoxyethanol and tetrasodium EDTA (ethylenediamenetraacetic acid tetrasodium salt) and fragrance. In other embodiments, the composition consists of water, synephrine, PEG-6 caprylic/capric glycerides, glycolic acid, at least one fragrance, propanediol, phenoxyethanol, sodium metabisulfite, panthenol, niacinamide, ethylhexylgylcerin, butylated hydroxytoluene, and tetrasodium EDTA (ethylenediamenetraacetic acid tetrasodium salt).

An example of one particular composition according to the present invention is a composition comprising about 10% to about 60% by weight of synephrine and about 5% to about 40% by weight of PEG-6 caprylic/capric glycerides. In some embodiments, the composition comprises about 30% to about 60% by weight of water, about 25% to about 45% by weight of synephrine, and about 5% to about 40% by weight of PEG-6 caprylic/capric glycerides. In some embodiments, the composition comprises about 40% to about 60% by weight of water, about 30% to about 50% by weight of synephrine, and about 8% to about 12% by weight of PEG-6 caprylic/capric glycerides.

In another example of a particular composition according to the present invention, the composition comprises about 30% to about 60% by weight of water, about 30% to about 45% by weight of synephrine, about 10% to about 20% by weight of glycolic acid, about 5% to about 15% by weight of PEG-6 caprylic/capric glycerides, about 4% to about 12% by weight of hydrochloric acid, about 1% to about 5% by weight of propanediol, about 0.4% to about 3% by weight of phenoxyethanol, about 0.1% to about 0.5% by weight of sodium metabisulfite, about 0.02% to about 0.5% by weight of panthenol, about 0.02% to about 0.5% by weight of niacinamide, about 0.04% to about 0.3% ethylhexylgylcerin, about 0.02% to about 0.5% by weight of butylated hydroxytoluene, and about 0.02% to about 0.5% by weight of tetrasodium EDTA (ethylenediamenetraacetic acid tetrasodium salt). In one or more embodiments, the composition may further comprise about 0.1% to about 2% by weight of a fragrance.

In yet another example of a particular composition according to the present invention, the composition comprises about 30% to about 50% by weight of water, about 28% to about 42% by weight of synephrine, about 8% to about 12% by weight of PEG-6 caprylic/capric glycerides, about 2% to about 4% by weight of propanediol, about 0.7% to about 1.1% by weight of phenoxyethanol ethylhexylglycerin, about 0.1% to about 0.5% by weight of sodium metabisulfite, about 0.01% to about 0.15% by weight of panthenol, about 0.01% to about 0.15% by weight of niacinamide, about 0.1% to about 2.0% phenoxyethanol ethylhexylgylcerin, about 0.05% to about 0.15% by weight of butylated hydroxytoluene, and about 0.05% to about 0.15% by weight of tetrasodium EDTA. In one or more embodiments, the composition may further comprise about 0.2% to about 0.8% by weight of a fragrance.

In an exemplary embodiment, the composition may comprise about 40-50% by weight of water, about 30-40% by weight of synephrine, and about 8-10% by weight of Acconon® CC-6 (PEG-6 caprylic/capric glycerides). The composition further may comprise about 3% by weight of propanediol, about 0.9% by weight of phenoxyethanol ethylhexylglycerin, about 0.5% by weight of a fragrance, about 0.3% by weight of sodium metabisulfite, about 0.1% by weight of panthenol, about 0.05% by weight of niacinamide, about 1.0% by weight of phenoxyethanol ethylhexylgylcerin, about 0.1% by weight of butylated hydroxytoluene, and about 0.1% by weight of tetrasodium EDTA (ethylenediamenetraacetic acid tetrasodium salt).

In some embodiments, the composition comprises water, synephrine, PEG-6 caprylic/capric glycerides, propanediol, phenoxyethanol ethylhexylgylcerin, one or more preservatives and one or more humectants. In one or more embodiments, the composition may further comprise a fragrance.

In some embodiments, the composition comprises water, R-(−)-synephrine, PEG-6 caprylic/capric glycerides, propanediol, phenoxyethanol, sodium metabisulfite, panthenol, niacinamide, phenoxyethanol ethylhexylgylcerin, butylated hydroxytoluene, and tetrasodium EDTA (ethylenediamenetraacetic acid tetrasodium salt). In one or more embodiments, the composition may further comprise a fragrance.

In some embodiments, the composition comprises water, R-(−)-synephrine, S-(+)-synephrine, PEG-6 caprylic/capric glycerides, propanediol, phenoxyethanol, sodium metabisulfite, panthenol, niacinamide, phenoxyethanol ethylhexylgylcerin, butylated hydroxytoluene, and tetrasodium EDTA (ethylenediamenetraacetic acid tetrasodium salt). In one or more embodiments, the composition may further comprise a fragrance and a surfactant, such as Tween 80, to aid in solubilizing the fragrance.

An example of one particular composition according to the present invention is a composition comprising about 30% to about 50% by weight of water, about 40% to about 70% by weight of synthetically produced synephrine, and about 5% to about 60% by weight of PEG-6 caprylic/capric glycerides. In some embodiments, the composition comprises about 40% to about 60% by weight of water, about 30% to about 60% by weight of synephrine HCl, and about 8% to about 20% by weight of PEG-6 caprylic/capric glycerides.

In some embodiments, the composition consists essentially of water, synephrine, PEG-6 caprylic/capric glycerides, at least one fragrance, propanediol, phenoxyethanol ethylhexylgylcerin, sodium metabisulfite, panthenol, niacinamide, and one or more preservatives such as without limitation butylated hydroxytoluene and tetrasodium EDTA (ethylenediamenetraacetic acid tetrasodium salt).

In some embodiments, the composition consists of water, synephrine, glycolic acid, PEG-6 caprylic/capric glycerides, hydrochloric acid, at least one fragrance, propanediol, phenoxyethanol, sodium metabisulfite, panthenol, niacinamide, ethylhexylgylcerin, butylated hydroxytoluene, and tetrasodium EDTA (ethylenediamenetraacetic acid tetrasodium salt).

Compositions Comprising Less Penetration Enhancer

In some embodiments, the composition comprises less penetration enhancer than the compositions discussed above. In such embodiments, the amount of synephrine is increased to accommodate the reduced penetration efficacy.

An example of one particular composition according to the present invention is a composition comprising about 20% to about 40% by weight of water, about 40% to about 60% by weight of synephrine, and about 3% to about 10% by weight of a penetration enhancer such as PEG-6 caprylic/capric glycerides. In some embodiments, the composition comprises about 30% to about 35% by weight of water, about 45% to about 55% by weight of synephrine, and about 3% to about 8% by weight of one or more penetration enhancers such as PEG-6 caprylic/capric glycerides.

Methods of Use

Embodiments of the compositions described herein may be used in methods of treating or preventing various conditions related to hair loss and/or hair shedding, and especially hair loss related to CIA or other forms of alopecia. In one embodiment, therapeutically effective amounts of the compositions described herein are applied topically to the scalp or other body surface containing hair follicles where prevention or treatment of hair loss is desired due to chemotherapy and/or radiotherapy treatment. Embodiments of the composition can be applied before, during, and/or after chemotherapy and/or radiotherapy treatment. Embodiments of composition can be applied multiple times. The same or different composition may be applied when multiple applications are used.

In at least one embodiment, a method of treatment can include applying an amount of a first composition to at least a portion of the scalp of a person before, during, and/or after chemotherapy and/or radiation therapy. The method can further include diagnosing response with a scalp perfusion test to determine if perfusion is reduced by a predetermined amount. The predetermined amount can be a reduction of perfusion of over 10%, over 20%, over 30%, over 40%, over 50%, over 60%, over 70%, over 80%, over 90%, etc. The predetermined amount can also be a reduction of perfusion within a range from 10-20%, 20-30%, 30-40%, 40-50%, etc. Other percent reductions or ranges of percent reductions can be used as the predetermined amount. If perfusion is reduced by the predetermined amount, the method can include applying a therapeutic effective amount of the first composition for treatment and/or prophylaxis of forms of alopecia. If perfusion is not reduced by the predetermined amount, the method can include applying a second composition to at least a portion of the scalp of the person. Diagnosing response with a scalp perfusion test may be performed again. If perfusion is reduced by the predetermined amount, the method can include applying a therapeutic effective amount of the second composition for treatment and/or prophylaxis of forms of alopecia. If perfusion is not reduced by the predetermined amount, the method can include applying a third composition to at least a portion of the scalp of the person. The first composition can include a concentration of the topical agents and/or modulating agents disclosed herein that is less than a concentration of the topical agents and/or modulating agents disclosed herein in the second composition. The second composition can include a concentration of the topical agents and/or modulating agents disclosed herein that is less than a concentration of the topical agents and/or modulating agents disclosed herein in the third composition.

In at least one embodiment, a method of treatment can include diagnosing response with laser Doppler or perfusion reduction (e.g., test at baseline, 15, 30, 60 minutes or a few points). Other tests for scalp perfusion can be used. These can include direct capillary pressure measurement, transcutaneous oxygen measurement, radionuclide techniques, temperature techniques (radiometric measurements, thermography, microwave radiometry, thermal clearance or conductivity measurements), ultrasound, dermofluorometry, laser Doppler flowmetry, photoplethysmography, capillary microscopy, other oxygenation techniques, other temperature techniques, etc.

Embodiments of the composition can be liquid solutions. The liquid solution may be applied directly to the scalp and rubbed into the scalp, or applied by spraying on with a delivery device such as a pump sprayer. Embodiments of the composition may further be combined with a shampoo or a conditioner or other hair care product to create a product that has more than one function.

In some embodiments, the compositions described herein are provided as a kit. A “kit” typically defines a package including at least one embodiment of the composition and another item that may be useful in its application, such as a comb, brush or other applicator, or with another hair care composition product, such as a shampoo, hair color/dye, hair oil or conditioner. For example, a kit may be a package containing at least one embodiment of the composition and a spray container or a dropper for administering the composition. In another embodiment, the kit can be a package containing (1) the an embodiment of the composition and (2) one or more of a shampoo, hairspray, conditioner, detangling solution, hair color, henna, or hair oil (such as without limitation coconut oil, jojoba oil, olive oil, baby oil, and black castor oil) and optionally (3) a pump spray container holding an embodiment of the composition or suitable to hold the composition. In another embodiment, the kit may include a container containing an embodiment of the composition and an applicator configured test a region of the scalp of a person for perfusion. Other embodiments of the kit can include a container containing an embodiment of the composition and a disposable device (e.g., a tip) that can be used in conjunction with an applicator, the applicator being configured to test a region of the scalp of a person for perfusion. The applicator in this embodiment may or may not be part of the kit.

### EXAMPLES

### Example 1: Extracting and Purifying R-(−)-Synephrine from Bitter Orange Extract

R-(−)-Synephrine was extracted from Citrus Aurantium in a first stage, and purified in a second stage.

To washed raw Citrus Aurantium (Citrus Aurantium L.), alcohol was added and used to extract synephrine from the raw material. The purity of the alcohol used for extraction was at least 80%. The extraction was repeated three times for about 2 hours each. The extraction formed an extract solution.

The extract solution was concentrated under a vacuum pressure of −0.08 mpa at a temperature of 60° C. The concentrated solution formed a first extractum.

The first extractum was agitated and dissolved in ethanol. The ethanol had a purity of at least about 98% ethanol. The extractum dissolved in the ethanol formed a first solution.

The first solution was applied to resin and eluted. A disused mobile phase was discarded.

Next, the eluent was concentrated. Similar to the step discussed above, the eluent was concentrated under a vacuum pressure of −0.08 mpa at a temperature of 60° C. The concentrated solution formed a second extractum.

The second extractum was dissolved in a second solution. The second solution with the dissolved second extractum had a liquid specific gravity of about 1.08.

The second solution was spray dried. The dried product comprised about 30% by weight of R-(−)-synephrine. The dried product of about 30% by weight of R-(−)-synephrine was further purified in a second stage. Starting materials containing less than 30% synephrine may be used and isolated and purified using the above procedures to yield a purified product of lower concentration.

In the second stage, the dried product of about 30% by weight of R-(−)-synephrine was agitated and dissolved in ethanol. The ethanol was at least about 98% ethanol. R-(−)-synephrine was crystallized from alcohol for about 12 hours. The solid phase was vacuum dried to a purity of at least about 98% by weight of R-(−)-synephrine. Purity of synephrine was measured using HPLC. Enantiomer concentrations can be determined using circular dichroism.

### Example 2—Alternative Second Stage

The steps were performed as in Example 1, except that in the second stage, the dried product of about 30% by weight of R-(−)-synephrine was agitated and dissolved in ethanol. The ethanol had a purity of at least about 98% ethanol. R-(−)-synephrine was crystallized from alcohol for about 12 hours. The solution phase passed through activated carbon for decoloration, producing a destaining solution. R-(−)-synephrine was crystallized from the destaining solution comprising alcohol for about 12 hours. The resulting crystal comprises at least about 95% by weight of R-(−)-synephrine.

### Example 3—Composition of Formula 00

TABLE 2

| | | % by | |
|---|---|---|---|
| Phase | Ingredient (Trade Name) | Weight | INCI Name |
| A | WATER | 32.95 | Water |
| A | HYDROCHLORIC | 7.56 | Hydrochloric Acid |
| | ACID | | |
| A | GLYCOLIC ACID (70% | 14.29 | Glycolic Acid |
| | Solution) | | |
| B | BITTER ORANGE | 30.00 | Citrus Aurantium Amara |
| | EXTRACT | | Extract |
| C | ACCONON CC-6 ® | 10.00 | PEG-6 Caprylic/Capric |
| | | | Glycerides |
| C | Fragrance | 0.50 | Fragrance |
| D | ZEMEA PROPANEDIOL | 3.00 | Propanediol |
| D | EUXYL PE 9010 (90%) | 0.90 | Phenoxyethanol |
| D | EUXYL PE 9010 (10%) | 0.10 | Ethylhexylglycerin |
| E | SODIUM | 0.30 | Sodium Metabisulfite |
| | METABISULFITE | | |
| E | dl PANTHENOL | 0.10 | Panthenol |
| E | NIACINAMIDE | 0.10 | Niacinamide |
| E | BUTYLATED | 0.10 | Butylated |
| | HYDROXYTOLUENE | | hydroxytoluene |
| E | TETRASODIUM EDTA | 0.10 | Tetrasodium EDTA |

The components were grouped into the phases listed in Table 2 above to organize the manufacturing process.

First, the components of Phase A were mixed together. The mixture was referred to as the batch.

Second, Phase B was slowly added to Phase A with mixing until all material was added and uniformly dissolved/dispersed in the batch. The pH was checked and adjusted to a pH of about 7.6. To adjust the pH, 5M NaOH was added to raise the pH or 5M HCL was added to lower the pH. The solution comprising Phase A and Phase B mixed together was clear or slightly hazy.

Third, the components of Phase C were mixed together. Next, Phase C was added to the batch and mixed until Phase C dissolved.

Fourth, the components of Phase D were mixed together. Next, Phase D was added to the batch and mixed until Phase D dissolved.

Fifth, each component of Phase E was added to the batch one at a time. The batch was mixed until uniform after the addition of each component in Phase E. The pH was checked and adjusted to a pH of about 7.6 by adding 5M NaOH to raise the pH or adding 5M HCL to lower the pH.

Sixth, the batch sat for at least 24 hours. After sitting, the batch was filtered through coarse filter paper.

Stability tests were performed on the composition of formula 00. Accelerated aging tests are useful in helping to determine expected formulation degradation over a long period of time by subjecting the product to elevated temperatures for a much shorter time period. Shelf-life data for a product can be extrapolated using the Arrhenius Equation, which relates chemical reaction rate (k) to absolute temperature (T). Samples of synephrine compositions are analyzed using a UV-visual spectrophotometer (Shamadzu, UV-1700). At least 5 separate serial dilutions are suggested to take the average of absorbance values. For synephrine, 273 nm is the wavelength chosen. Absorption data can be used to determine percent mass loss over time period tested. This data can be extrapolated to determine long-term stability of the sample.

The results of the accelerated aging tests are reported in the following table:

TABLE 3

| Day (room temperature | Absolute | |
|---|---|---|
| equivalent) | intensity | % activity of synephrine |
| 0 | 4279 | 100% |
| 37 | 3389 | 79% |
| 91 | 3312 | 77% |
| 128 | 3300 | 77% |

### Example 4—Composition of Formula 01

TABLE 4

| | | % by | |
|---|---|---|---|
| Phase | Ingredient (Trade Name) | Weight | INCI Name |
| A | WATER | 47.15 | Water |
| A | TETRASODIUM EDTA | 0.10 | Tetrasodium EDTA |
| A | SODIUM | 0.30 | sodium |
| | METABISULFITE | | metabisulfite |
| A | SYNEPHRINE | 37.00 | P-synephrine HCl |
| A | dl PANTHENOL | 0.05 | Panthenol |
| A | NIACINAMIDE | 0.05 | Niacinamide |
| B | ACCONON CC-6 ® | 10.00 | PEG-6 Caprylic/Capric |
| | | | Glycerides |
| B | Fragrance | 0.525 | Fragrance |
| B | Polysorbate 80 | 1.00 | Polysorbate 80 |
| C | BHT | 0.10 | Butylated hydroxytoluene |
| D | ZEMEA PROPANEDIOL | 3.00 | 1,3-Propanediol |
| D | EUXYL PE 9010 (90%) | 1.00 | Phenoxyethanol |
| | | | Ethylhexylglycerin |

To produce the composition of Example 4, the components were grouped into the phases listed in the table above. First, in step 1, the components of Phase A were mixed together until uniformly dispersed or dissolved. The mixture will be referred to as the batch. Second, the components of Phase B were mixed together with heat until all material is added and uniformly dissolved/dispersed. Third, phase C was added to B and mixed until uniform. Fourth, in step 4 Phase D was added to the mixture of B and C with mixing. Fifth, the step 1 and step four solutions were mixed together with heat (45 degrees C.) until completely dissolved. Sixth, the resulting mixture was cooled to room temperature, pH was adjusted to about 4.9 to 5.3 with NaOH or HCl and the product was allowed to sit for at least one day, before subjecting to filtration over coarse filter paper.

### Example 5—Composition of Formula 04

TABLE 5

| | % by | |
|---|---|---|
| Ingredient (Trade Name) | Weight | INCI Name |
| WATER | 56.15 | Water |
| TETRASODIUM EDTA | 0.10 | Tetrasodium EDTA |
| SODIUM | 0.30 | sodium |
| METABISULFITE | | metabisulfite |
| SYNEPHRINE | 37.00 | P-synephrine HCl |
| dl PANTHENOL | 0.05 | Panthenol |
| NIACINAMIDE | 0.05 | Niacinamide |
| Di(ethylene glycol) ethyl | 1.00 | Di(ethylene glycol) ethyl |
| ether | | ether |
| Fragrance | 0.25 | Fragrance |
| Polysorbate 80 | 1.00 | Polysorbate 80 |
| BHT | 0.10 | Butylated hydroxytoluene |
| ZEMEA PROPANEDIOL | 3.00 | 1,3-Propanediol |
| EUXYL PE 9010 (90%) | 1.00 | Phenoxyethanol |
| | | Ethylhexylglycerin |

The composition of Example 5 (formula 04) was prepared analogously to the composition of Example 4.

### Example 6

Safety Study

Synephrine has been studied extensively in its oral form and has not been shown to induce significant cardiac or hemodynamic changes (Stohs 2011). Synephrine is an isomer of phenylephrine, different only in the position of the phenolic hydroxyl group; phenylephrine has a meta-hydroxyl, while synephrine has a para-hydroxyl. Synephrine is a weak A1AR agonist, exhibiting a potency approximately 150 fold less that of phenylephrine (Brown 1988). As such, a higher concentration may be needed to observe the same A1AR activation on the scalp. As noted above, the higher concentration of the active can facilitate penetration through the stratum corneum.

A study was conducted to determine the safety of a topical solution containing 37% synephrine HCl (the composition of formula 01). 10 mL of the topical solution was applied to the entire scalp and rubbed thoroughly at a base line. An additional 10 mL was applied after 2 hours. Blood pressure (BP), heart rate (HR) and EKG measurements were taken using a wearable holter monitor. In the study, the composition was applied to the entire scalp and no significant cardiac or hemodynamic changes occurred.

In total, 40 female subjects ages 18-65 in good health were enrolled. 30 of the subjects received the active solution and 10 received the placebo (vehicle only). To evaluate the effect of the composition on systemic hemodynamic parameters, a holter monitor was placed on each subject. At baseline, BP and HR were recorded. BP, HR, and EKG data were collected over a 24-hour period via the holter monitor. In the 24 hours following application of the solution, none of the subjects exhibited any treatment related adverse events (e.g., no significant changes in hemodynamic parameters and no systemic effects). No significant changes in BP and/or HR were observed for any of the subjects. EKG parameters (e.g., ST segments, arrhythmias, etc.) were normal throughout the 24 hour period.

These data demonstrates that activation of A1AR is not likely outside of the local application site, which may be due to the low A1AR binding affinity of synephrine. Without being limited by theory in any way, the results with synephrine may be due to the low A1AR binding affinity (Ma 2010, Hibino 2009) of synephrine resulting in no noticeable systemic effects once diluted outside of the local application site.

Efficacy can also be improved. Details of an efficacy test that was conducted is presented blow.

Efficacy Study

A pilot study was conducted to determine tissue induced hypoxia following application of a topical solution containing 37% synephrine HCl (e.g., the composition of formula 01). 10 mL of the topical solution was applied to the entire scalp and rubbed thoroughly at a base line. In total, ten female subjects ages 18-65 in good health were enrolled. Five of the subjects received the active solution and five received the placebo (vehicle only). Laser Doppler velocimetry was used to measure changes in scalp tissue oxygenation at the baseline, 30 minutes, 1 hour, and 2 hours post application of the solution. In four of the five subjects receiving the active synephrine HCl, scalp oxygenation was reduced by up to 88%, with an average of 59% reduction compared to the baseline. In the control group, no scalp oxygenation change was observed.

### Example 7

Assessment of Successful Hair Preservation

Successful hair preservation can be assessed using the Common Terminology Criteria for Adverse Events (CTCAE) version 4.0 scale (see Table 6) at the end of 4 cycles of chemotherapy. In addition, or in the alternative, global photographs can be collected after each chemotherapy cycle and evaluated by an independent physician.

TABLE 6
Skin and subcutaneous tissue disorders

| Adverse | Grade | | | | |
|---|---|---|---|---|---|
| Event | 1 | 2 | 3 | 4 | 5 |
| Alopecia | Hair loss of <50% of normal | Hair loss of >=50% normal | — | — | — |
| | for that individual that is not | for that individual that is | | | |
| | obvious from a distance but | readily apparent to others; a | | | |
| | only on close inspection; a | wig or hair piece is | | | |
| | different hair style may be | necessary if the patient | | | |
| | required to cover the hair loss | desires to completely | | | |
| | but it does not require a wig or | camouflage the hair loss; | | | |
| | hair piece to camouflage | associated with | | | |
| | | psychosocial impact | | | |

Definition: A disorder characterized by a decrease in density of hair compared to normal for a given individual at a given age and body location.

Successful hair prevention trials will be conducted using an embodiment of the composition containing synephrine. Successful hair preservation will be assessed using the CTCAE version 4.0 scale, where grade 0 [e.g., no hair loss] or grade 1 [e.g., <50% hair loss not requiring a wig] will be considered successful. This will be assessed at the conclusion of 4 cycles of chemotherapy. Scalp blood perfusion will be measured with a laser Doppler velocimeter before and after application of the composition [Baseline, 60 minutes].

The total number of subjects will be 80. The duration of the study is 8-12 weeks (between 2-3 weeks per treatment cycle). The study will be a double-blinded placebo controlled study. A total of 80 subjects will be enrolled. 30 subjects with breast cancer will be administered the composition and 10 subjects with breast cancer will be administered the placebo vehicle solution. An additional 30 subjects with solid organ cancer will be administered the composition and 10 subjects with solid organ cancer will be administered the placebo vehicle solution.

Study Phase I: Laser Doppler Velocimetry
1. Each potential subject will be screened for the exclusion and inclusion criteria.
   Inclusion Criteria
      Stage I or II breast cancer
      Stage I or II solid organ cancer
      Scheduled but not begun, at least 4 cycles of Taxane and/or Anthracycline-based chemotherapy.
      Ages 18-65
   Exclusion Criteria
      Subjects with uncontrolled hypertension
      Subjects diagnosed with pattern hair loss or with other hair loss in conjunction with female pattern hair loss
      Folliculitis
      Scalp psoriasis
      Seborrheic dermatitis
      Inflammatory scalp conditions such as lichen planopillaris
      Subjects wearing wigs prior to chemotherapy
2. BP of each subject will be measure and recorded.
3. A target area of 1 cm² on the top of the scalp will be marked with a surgical pen.
4. Blood perfusion of the subject within the target area will be measured and recorded.
5. 5 mL of the composition will be applied to the entire scalp and massage the solution into subject's scalp.
6. An additional 5 mL of the composition will be applied to the entire scalp again and the solution will be massaged into subject's scalp again.
7. After 60 minutes, the scalp blood perfusion of the subjects within the target area will be measured.
8. The BP of each subject will be measured and recorded.

Study Phase II: Chemotherapy Treatment
1. 10-15 minutes prior to each chemotherapy session, 5 mL of the composition will be applied to the entire scalp and it will be massaged into subject's scalp.
2. An additional 5 mL of the composition will be applied to the entire scalp again and massaged into subject's scalp again.
3. The subject will complete a normal chemotherapy treatment session.
4. At the end of each chemotherapy session, 5 mL of the composition will be applied to the entire scalp and massaged into subject's scalp.
5. An additional 5 mL of the composition will be applied to the entire scalp and massaged into subject's scalp again.
6. After completion of each chemotherapy cycle (2-3 weeks), the subject's hair loss will be documented using global photography.
7. A total of 4 cycles of chemotherapy will be documented.

Clinical assessment of efficacy can be performed by assessing scalp blood perfusion after 60 minutes of applying the composition versus the baseline. Scalp Blood perfusion will be measured with laser Doppler velocimetry. Reduction in scalp blood perfusion will be expressed as percent reduction from the baseline, using the CTCAE version 4.0 scale at the end of 4 cycles of chemotherapy. Global photographs will be collected after each chemotherapy cycle and evaluated by an independent physician.

Each test site will be assessed for any potential irritation or sensitisation from the application of the composition, which may include any complaints of adverse events reported by the subjects. The heart rate and blood pressure of each subject using the composition will be closely monitored.

Some embodiments can involve use of a modified CTCAE scale. This can include use of the Dean scale. The following hair loss indicators can be used for the Dean scale

Grade: 0 indicated no hair loss
Grade 1: >0 up to 25% hair loss
Grade 2: >25 up to 50% hair loss
Grade 3: >50 up to 75% hair loss
Grade 4: >75% hair loss

Treatment using the methods disclosed herein can be deemed successful if the maximum Dean score is <2 four weeks after the last chemotherapy session. The scoring can be performed by the patient, a physician, or an independent expert based on photography.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

All patents and other publications, including literature references, issued patents, published patent applications, and co-pending patent applications, cited throughout this application are expressly incorporated herein by reference in their entireties.

### REFERENCES

Balsari, A. L.; Morelli, D.; Menard, S.; Veronesi, U. & Colnagho, M. I. (1994) Protection against doxorubicin-induced alopecia in rats by liposome-entrapped monoclonal antibodies. The FASEB Journal Vol. 8, No. 2, (February 1994), pp. 226-230.
Batchelor, D. (2001). Hair and cancer chemotherapy: consequences and nursing care—a literature study. European Journal of Cancer Care Vol. 10, No. 3, (September 2001), pp. 147-163.
C. M. Brown, J. C. McGrath, J. M. Midgley, A. G. Muir, J. W. O'Brien et al., “Activities of octapamine and synephrine stereoisomers on alpha-adrenoreceptors,” British Journal of Pharmacology, vol. 93, pp. 417-429, 1988,
Conklin, K. A. (2004) Chemotherapy-associated oxidative stress: impact on chemotherapeutic effectiveness. Integrative Cancer Therapies Vol. 3, No. 4, (December 2004), pp. 294-300.
Davis, S. T.; Benson, B. G.; Bramson, H. N.; Chapman, D. E.; Dickerson, S. H.; Dold, K. M.; Eberwein, D. J.; Edelstein, M.; Frye, S. V.; Gampe Jr., R. T.; Griffin, R. J.; Harris, P. A.; Hassel, A. M.; Holmes, W. D.; Hunter, R. N.; Knick, V. B.; Lackey, K.; Lovejoy, B.; Luzzio, M. J.; Murray, D.; Parker, P.; Rocque, W. J.; Shewchuk, L.; Veal, J. M.; Walker, D. H. & Kuyper, L. F. (2001) Prevention of chemotherapy-induced alopecia in rats by CDK inhibitors. Sciences Vol. 291, No. 5501, (January 2001), pp. 134-137.
FDA Press Release: https://www.fda.gov/newsevents/newsroom/pressannouncements/ucm476216.htm, accessed on Jul. 31, 2017.
G. Ma, S. A. Bavadekar, B. T. Schaneberg, I. A. Khan, and D. R. Feller, “Effects of synephrine and β-phenethylamine on human α-adrenoceptor subtypes,” Planta Medica, vol. 76, no. 10, pp. 981-986, 2010.
Huang L, Wu Q, Li Y, Wang Y, Bi H. PXR-Mediated Upregulation of CYP3A Expression by Herb Compound Praeruptorin C from Peucedanum praeruptorum Dunn. Evid Based Complement Alternat Med. 2013; 2013: 156574.
Hussein, A. M.; Stuart, A. & Peters, W. P. (1995) Protection against chemotherapy-induced alopecia by cyclosporine A in the newborn rat animal model. Dermatology Vol. 190, No. 3, (1995), pp. 192-196.
Jimenez, J. J. & Yunis, A. A. (1996) Vitamin D3 and chemotherapy-induced alopecia. Nutrition Vol. 12, No. 6, (June 1996), pp. 448-449.
McGarvey E L, Baum L D, Pinkerton R C, et al. Psychological sequelae and alopecia among women with cancer. Cancer Pract 9(6):283-9 (2001 November-December).
Nangia J, Wang T, Osborne C, Niravath P, Otte K, Papish S, Holmes F, Abraham J, Lacouture M, Courtright J, Paxman R, Rude M, Hilsenbeck S, Osborne C K, Rimawi M. Effect of a Scalp Cooling Device on Alopecia in Women Undergoing Chemotherapy for Breast Cancer: The SCALP Randomized Clinical Trial. JAMA. 2017 Feb. 14; 317(6): 596-605.
Schilli M B, Paus R, Menrad A. Reduction of intrafollicular apoptosis in chemotherapy-induced alopecia by topical calcitriol-analogs. J Invest Dermatol. 1998 October; 111(4):598-604.
Soref C M, Fahl W E. Int J Cancer. A new strategy to prevent chemotherapy and radiotherapy-induced alopecia using topically applied vasoconstrictor. 2015 Jan. 1; 136(1): 195-203.
Stohs S J, Preuss H G, Shara M. A review of the receptor-binding properties of p-synephrine as related to its pharmacological effects. Oxid Med Cell Longev. 2011; 2011:482973. doi: 10.1155/2011/482973. Epub 2011 Aug. 1.
T. Hibino, M. Yuzurihara, Y. Kase, and A. Takeda, “Synephrine, a component of Evodiae fructus, constricts isolated aorta via adrenergic and serotonergic receptors,” Journal of Pharmacological Sciences, vol. 111, no. 1, pp. 73-81, 2009.
Trueb R M. Chemotherapy-induced alopecia. Semin Cutan Med Surg 28(1): 11-4 (2009 March).
Tsuda, T.; Ohmori, Y.; Muramatsu, H.; Hosaka, Y.; Takigushi, K.; Saitoh, F.; Kato, K.;
Nakayama, K.; Nakamura, N.; Nagata, S. & Mochizuki, H. (2001) Inhibitory effect of M50054, a novel inhibitor of apoptosis, on anti-Fas-antibody-induced hepatitis and chemotherapy-induced alopecia. European Journal of Pharmacology Vol. 433, No. 1, (December 2001), pp. 43337-43345.
Wang, J.; Lu, Z. & Au, J. L. S. (2006) Protection against chemotherapy-induced alopecia. Pharmaceutical Research Vol. 23, No. 11, (November 2006), pp. 2505-2514.
de Weger V A, Beijnen J H, Schellens J H. Cellular and clinical pharmacology of the taxanes docetaxel and paclitaxel—a review. Anticancer Drugs. 2014 May; 25(5):488-94.

What is claimed is:

1. A method of treatment and/or prophylaxis of forms of alopecia comprising applying a composition topically to a scalp of a person before, during, and/or after undergoing chemotherapy and/or radiation therapy, wherein the composition comprises a therapeutically effective amount of a weak acting alpha-1 adrenergic receptor agonist.

2. The method of claim 1, wherein by applying the composition to the person hypoxia in local tissue is induced.

3. The method of claim 1, wherein by applying the composition to the person amounts of chemotherapeutic agents and/or reactive oxygen species agents delivered to a hair follicle is limited.

4. The method of claim 1, wherein the composition is applied to maintain a therapeutically effective amount for a prolonged period of time, the prolonged period of time being within a range from 1 hour to 48 hours.

5. The method of claim 1, wherein the weak acting agonist to alpha-1 adrenergic receptor is at least one of a selective alpha-1 adrenergic receptor agonist and an agonist having a low affinity for adrenergic receptors that are not alpha-1 adrenergic receptors.

6. The method of claim 1, wherein the weak acting agonist to alpha-1 adrenergic receptors is synephrine.

7. The method of claim 1, wherein the composition further comprises a penetration enhancer.

8. The method of claim 7, wherein the weak acting agonist to alpha-1 adrenergic receptors is synephrine and the penetration enhancer is pegylated caprylic/capric glycerides.

9. The method of claim 1, wherein the composition comprises about 30% to about 50% by weight of water, about 25% to about 40% by weight of synephrine, and about 5% to about 30% by weight of PEG-6 caprylic/capric glycerides.

10. The method of claim 9, wherein the form of alopecia is chemotherapy induced alopecia and the chemotherapy is Taxane and/or Anthracycline-based chemotherapy.

11. The method of claim 10, wherein the chemotherapy is used to treat stage I and/or stage II breast cancer patients.

12. A method of treatment and/or prophylaxis of forms of alopecia comprising applying a composition to a scalp of a person before, during, and/or after undergoing chemotherapy and/or radiation therapy, the composition comprising a therapeutically effective amount of a weak acting alpha-1 adrenergic receptor agonist and a modulating agent, wherein the modulating agent is configured to modulate deposition, mode of action, and/or metabolism of a chemotherapeutic agent and/or a reactive oxygen species agent.

13. The method of claim 12, wherein the form of alopecia is chemotherapy induced alopecia and the chemotherapy is Taxane and/or Anthracycline-based chemotherapy.

14. The method of claim 13, wherein the chemotherapy is used to treat stage I and/or stage II breast cancer patients.

15. A method of treatment and/or prophylaxis of forms of alopecia comprising applying a composition to a scalp of a person before, during, and/or after undergoing chemotherapy and/or radiation therapy, the composition comprising a therapeutically effective amount of a topical agent, wherein the topical agent is configured to up-regulate CYP3A.

16. The method of claim 15, wherein the form of alopecia is chemotherapy induced alopecia and the chemotherapy is Taxane and/or Anthracycline-based chemotherapy.

17. The method of claim 16, wherein the chemotherapy is used to treat stage I and/or stage II breast cancer patients.

18. A method of treatment and/or prophylaxis of forms of alopecia comprising applying a composition to a scalp of a person before, during, and/or after undergoing chemotherapy and/or radiation therapy, the composition comprising a therapeutically effective amount of a weak acting alpha-1 adrenergic receptor agonist and a modulating agent, the modulating agent configured to up-regulate CYP3A.

19. The method of claim 18, wherein the form of alopecia is chemotherapy induced alopecia and the chemotherapy is Taxane and/or Anthracycline-based chemotherapy.

20. The method of claim 19, wherein the chemotherapy is used to treat stage I and/or stage II breast cancer patients.

21. A kit for treatment and/or prophylaxis of forms of alopecia, comprising:
a container containing a weak acting alpha-1 adrenergic receptor agonist; and
at least one of:
an applicator configured to test a region of the scalp of a person for perfusion; and
a disposable tip configured for use with a perfusion testing device.

22. A kit for treatment and/or prophylaxis of forms of alopecia, comprising:
a container containing a weak acting alpha-1 adrenergic receptor agonist and a modulating agent, wherein the modulating agent is configured to modulate deposition, mode of action, and/or metabolism of a chemotherapeutic agent and/or a reactive oxygen species agent; and
at least one of:
an applicator configured to test a region of the scalp of a person for perfusion; and
a disposable tip configured for use with a perfusion testing device.

23. A kit for treatment and/or prophylaxis of forms of alopecia, comprising:
a container containing a topical agent, wherein the topical agent is configured to up-regulate CYP3A; and
at least one of:
an applicator configured to test a region of the scalp of a person for perfusion; and
a disposable tip configured for use with a perfusion testing device.

24. A kit for treatment and/or prophylaxis of forms of alopecia, comprising:
a container containing a weak acting alpha-1 adrenergic receptor agonist and a modulating agent, wherein the modulating agent is configured to up-regulate CYP3A; and
at least one of:
an applicator configured to test a region of the scalp of a person for perfusion; and
a disposable tip configured for use with a perfusion testing device.

25. A hair care product for treatment and/or prophylaxis of forms of alopecia, comprising a pump spray container containing a weak acting A1AR agonist.

26. A hair care product for treatment and/or prophylaxis of forms of alopecia, comprising a pump spray container containing a weak acting alpha-1 adrenergic receptor agonist and a modulating agent, wherein the modulating agent is configured to modulate deposition, mode of action, and/or metabolism of a chemotherapeutic agent and/or a reactive oxygen species agent.

27. A hair care product for treatment and/or prophylaxis of forms of alopecia, comprising a pump spray container containing a topical agent, wherein the topical agent is configured to up-regulate CYP3A.

28. A hair care product for treatment and/or prophylaxis of forms of alopecia, comprising a pump spray container containing a weak acting alpha-1 adrenergic receptor agonist and a modulating agent, wherein the modulating agent is configured to up-regulate CYP3A.

29. A method of reducing and/or inhibiting perfusion of an anti-cancer agent into noncancerous organ or tissue or development of forms of alopecia in a person comprising applying a therapeutically effective amount of a composition to the scalp of the person before, during, and/or after undergoing cancer treatment, the composition comprising about 30-40% synephrine by weight, water, PEG-6 caprylic/capric glycerides, and polysorbate.

30. The method of claim 29, wherein the composition further comprises a preservative, niacinamide, panthenol, sodium metabisulfite, phenoxyethanol ethylhexylglycerin and 1,3-propanediol.

31. A method of treatment and/or prophylaxis of forms of alopecia, comprising:
applying an amount of a first composition to at least a portion of a scalp of a person before, during, and/or after chemotherapy and/or radiation therapy; and
diagnosing response with a scalp perfusion test to determine if perfusion is reduced by a predetermined amount, wherein:
if perfusion is reduced by the predetermined amount, applying a therapeutic effective amount of the first composition for treatment and/or prophylaxis of forms of alopecia;
if perfusion is not reduced by the predetermined amount, applying a second composition to at least a portion of the scalp of the person;
diagnosing response with a scalp perfusion test to determine if perfusion is reduced by a predetermined amount after application of the second composition;
if perfusion is reduced by the predetermined amount, then applying a therapeutic effective amount of the second composition for treatment and/or prophylaxis of forms of alopecia;
wherein the first composition comprises a concentration of a weak acting alpha-1 adrenergic receptor agonist that is less than a concentration of the weak acting alpha-1 adrenergic receptor agonist in the second composition.

32. A method of treatment and/or prophylaxis of forms of alopecia, comprising:
applying an amount of a first composition to at least a portion of a scalp of a person before, during, and/or after chemotherapy and/or radiation therapy; and
diagnosing response with a scalp perfusion test to determine if perfusion is reduced by a predetermined amount, wherein:
if perfusion is reduced by the predetermined amount, applying a therapeutic effective amount of the first composition for treatment and/or prophylaxis of forms of alopecia;
if perfusion is not reduced by the predetermined amount, applying a second composition to at least a portion of the scalp of the person;
diagnosing response with a scalp perfusion test to determine if perfusion is reduced by a predetermined amount after application of the second composition;
if perfusion is reduced by the predetermined amount, then applying a therapeutic effective amount of the second composition for treatment and/or prophylaxis of forms of alopecia;
wherein the first composition comprises a concentration of a weak acting alpha-1 adrenergic receptor agonist and a modulating agent that is less than a concentration of the weak acting alpha-1 adrenergic receptor agonist and the modulating agent in the second composition;
wherein the modulating agent is configured to modulate deposition, mode of action, and/or metabolism of a chemotherapeutic agent and/or a reactive oxygen species agent.

33. A method of treatment and/or prophylaxis of forms of alopecia, comprising:
applying an amount of a first composition to at least a portion of a scalp of a person before, during, and/or after chemotherapy and/or radiation therapy; and
diagnosing response with a scalp perfusion test to determine if perfusion is reduced by a predetermined amount, wherein:
if perfusion is reduced by the predetermined amount, applying a therapeutic effective amount of the first composition for treatment and/or prophylaxis of forms of alopecia;
if perfusion is not reduced by the predetermined amount, applying a second composition to at least a portion of the scalp of the person;
diagnosing response with a scalp perfusion test to determine if perfusion is reduced by a predetermined amount after application of the second composition;
if perfusion is reduced by the predetermined amount, then applying a therapeutic effective amount of the second composition for treatment and/or prophylaxis of forms of alopecia;
wherein the first composition comprises a concentration of a topical agent that is less than a concentration of the topical agent in the second composition;
wherein the topical agent is configured to up-regulate CYP3A.

34. A method of treatment and/or prophylaxis of forms of alopecia, comprising:
applying an amount of a first composition to at least a portion of a scalp of a person before, during, and/or after chemotherapy and/or radiation therapy; and
diagnosing response with a scalp perfusion test to determine if perfusion is reduced by a predetermined amount, wherein:
if perfusion is reduced by the predetermined amount, applying a therapeutic effective amount of the first composition for treatment and/or prophylaxis of forms of alopecia;
if perfusion is not reduced by the predetermined amount, applying a second composition to at least a portion of the scalp of the person;
diagnosing response with a scalp perfusion test to determine if perfusion is reduced by a predetermined amount after application of the second composition;
if perfusion is reduced by the predetermined amount, then applying a therapeutic effective amount of the second composition for treatment and/or prophylaxis of forms of alopecia;
wherein the first composition comprises a concentration of a weak acting alpha-1 adrenergic receptor agonist and a modulating agent that is less than a concentration of the weak acting alpha-1 adrenergic receptor agonist and the modulating agent in the second composition;
wherein the modulating agent is configured to up-regulate CYP3A.
